(19) United States
(12) Patent Application Publication
(10) Pub. No.: US 2003/0212440 A1
(43) Pub. Date: Nov. 13, 2003

(54) Method and system for modulating the vagus nerve (10th cranial nerve) using modulated electrical pulses with an inductively coupled stimulation system

(76) Inventor: Birinder R. Boveja, Milwaukee, WI (US)

Correspondence Address: Angely Widhany, P O Box 210095, Milwaukee, WI, 53221, US

(21) Appl. No.: 10/196,533

(22) Filed: Jul. 16, 2002

Related U.S. Application Data
(63) Continuation-in-part of application No. 10/142,298, filed on May 9, 2002.

(51) Int. Cl. A61N 1/08

(52) U.S. Cl. 607/46

(57) ABSTRACT

A method and system for afferent neuromodulation of the vagus nerve for providing stimulation therapy, comprises a transmitter and an implanted stimulus receiver. The circuitry of the transmitter is capable of generating modulated high frequency pulses for delivering virtually any form, any sequence, for any time interval for various therapy application. The implanted stimulus receiver, which is in electrical connection with the vagus nerve, may be a passive or an active device. In one embodiment the implanted stimulus receiver may be a “hybrid device” which is both inductively coupled, as well as, having a power source. Therapy is provided via pre-determined program or may be “custom” adjusted for the patient. In one embodiment the external transmitter comprises a wireless communication telemetry module, whereby therapy programs may be adjusted remotely by the physician via the wireless internet.

This is a Continuation-in-Part application claiming priority from pending prior application Ser. No. 10/142,298 filed May 09, 2002, the prior application being incorporated herein by reference.

### FIELD OF INVENTION

The present invention relates to neuromodulation, more specifically neuromodulation of the vagus nerve using modulated high frequency electrical pulses with an inductively coupled system.

### BACKGROUND

The 10^{th} cranial nerve or the vagus nerve plays a role in mediating afferent information from visceral organs to the brain. The vagus nerve arises directly from the brain, but unlike the other cranial nerves extends well beyond the head. At its farthest extension it reaches the lower parts of the intestines. The vagus nerve provides an easily accessible, peripheral route to modulate central nervous system (CNS) function. Observations on the profound effect of electrical stimulation of the vagus nerve on central nervous system (CNS) activity extends back to the 1930's.

The present invention is primarily directed to a method and system for selective electrical stimulation or neuromodulation of vagus nerve, for providing adjunct therapy for neurological and neuropsychiatric disorders such as epilepsy, depression, anxiety disorders, neurogenic pain, compulsive eating disorders, obesity, Alzheimer's disease and the like.

In the human body there are two vagal nerves (VN), the right VN and the left VN. Each vagus nerve is encased in the carotid sheath along with the carotid artery and jugular vein. The innervation of the right and left vagus nerves is different. The innervation of the right vagus nerve is such that stimulating it results in profound bradycardia (slowing of the heart rate). The left vagus nerve has some innervation to the heart, but mostly innervates the visceral organs such as the gastrointestinal tract. It is known that stimulation of the left vagus nerve does not cause substantial slowing of the heart rate or cause any other significant deleterious side effects.

### Background of Neuromodulation

One of the fundamental features of the nervous system is its ability to generate and conduct electrical impulses. Most nerves in the human body are composed of thousands of fibers of different sizes. This is shown schematically in FIG. 1. The different sizes of nerve fibers, which carry signals to and from the brain, are designated by groups A, B, and C. The vagus nerve, for example, may have approximately 100,000 fibers of the three different types, each carrying signals. Each axon or fiber of that nerve conducts only in one direction, in normal circumstances. In the vagus nerve sensory fibers outnumber parasympathetic fibers four to one.

In a cross section of peripheral nerve it is seen that the diameter of individual fibers vary substantially, as is also shown schematically in FIG. 2. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated.

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

Nerve cells have membranes that are composed of lipids and proteins (shown schematically in FIGS. 3A and 3B), and have unique properties of excitability such that an adequate disturbance of the cell's resting potential can trigger a sudden change in the membrane conductance. Under resting conditions, the inside of the nerve cell is approximately −90 mV relative to the outside. The electrical signaling capabilities of neurons are based on ionic concentration gradients between the intracellular and extracellular compartments. The cell membrane is a complex of a bilayer of lipid molecules with an assortment of protein molecules embedded in it (FIG. 3A), separating these two compartments. Electrical balance is provided by concentration gradients which are maintained by a combination of selective permeability characteristics and active pumping mechanism.

The lipid component of the membrane is a double sheet of phospholipids, elongated molecules with polar groups at one end and the fatty acid chains at the other. The ions that carry the currents used for neuronal signaling are among these water-soluble substances, so the lipid bilayer is also an insulator, across which membrane potentials develop. In biophysical terms, the lipid bilayer is not permeable to ions. In electrical terms, it functions as a capacitor, able to store charges of opposite sign that are attracted to each other but unable to cross the membrane. Embedded in the lipid bilayer is a large assortment of proteins. These are proteins that regulate the passage of ions into or out of the cell. Certain membrane-spanning proteins allow selected ions to flow down electrical or concentration gradients or by pumping them across.

These membrane-spanning proteins consist of several subunits surrounding a central aqueous pore (shown in FIG. 3B). Ions whose size and charge “fit” the pore can diffuse through it, allowing these proteins to serve as ion channels. Hence, unlike the lipid bilayer, ion channels have an appreciable permeability (or conductance) to at least some ions. In electrical terms, they function as resistors, allowing a predicable amount of current flow in response to a voltage across them.

A nerve cell can be excited by increasing the electrical charge within the neuron, thus increasing the membrane potential inside the nerve with respect to the surrounding extracellular fluid. As shown in FIG. 4, stimuli 1 and 2 are subthreshold, and do not induce a response. Stimulus 3 exceeds a threshold value and induces an action potential (AP) which will be propagated. The threshold stimulus intensity is defined as that value at which the net inward current (which is largely determined by Sodium ions) is just greater than the net outward current (which is largely carried by Potassium ions), and is typically around −55 mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials, which are defined as a single electrical impulse passing down an axon. This action potential (nerve impulse or spike) is an “all or nothing” phenomenon, that is to say once the threshold stimulus intensity is reached, an action potential will be generated.

FIG. 5A illustrates a segment of the surface of the membrane of an excitable cell. Metabolic activity maintains ionic gradients across the membrane, resulting in a high concentration of potassium (K⁺) ions inside the cell and a high concentration of sodium (Na⁺) ions in the extracellular environment. The net result of the ionic gradient is a transmembrane potential that is largely dependent on the K⁺ gradient. Typically in nerve cells, the resting membrane potential (RMP) is slightly less than 90 mV, with the outside being positive with respect to inside.

To stimulate an excitable cell, it is only necessary to reduce the transmembrane potential by a critical amount. When the membrane potential is reduced by an amount ΔV, reaching the critical or threshold potential (TP); Which is shown in FIG. 5B. When the threshold potential (TP) is reached, a regenerative process takes place: sodium ions enter the cell, potassium ions exit the cell, and the transmembrane potential falls to zero (depolarizes), reverses slightly, and then recovers or repolarizes to the resting membrane potential (RMP).

For a stimulus to be effective in producing an excitation, it must have an abrupt onset, be intense enough, and last long enough. These facts can be drawn together by considering the delivery of a suddenly rising cathodal constant-current stimulus of duration d to the cell membrane as shown in FIG. 5B.

Cell membranes can be reasonably well represented by a capacitance C, shunted by a resistance R as shown by a simplified electrical model in diagram 5C, and shown in a more realistic electrical model in FIG. 6, where neuronal process is divided into unit lengths, which is represented in an electrical equivalent circuit. Each unit length of the process is a circuit with its own membrane resistance (rₘ), membrane capacitance (cₘ), and axonal resistance (rₐ).

When the stimulation pulse is strong enough, an action potential will be generated and propagated. As shown in FIG. 7, the action potential is traveling from right to left. Immediately after the spike of the action potential there is a refractory period when the neuron is either unexcitable (absolute refractory period) or only activated to sub-maximal responses by supra-threshold stimuli (relative refractory period). The absolute refractory period occurs at the time of maximal Sodium channel inactivation while the relative refractory period occurs at a later time when most of the Na⁺ channels have returned to their resting state by the voltage activated K⁺ current. The refractory period has two important implications for action potential generation and conduction. First, action potentials can be conducted only in one direction, away from the site of its generation, and secondly, they can be generated only up to certain limiting frequencies.

A single electrical impulse passing down an axon is shown schematically in FIG. 8. The top portion of the figure (A) shows conduction over mylinated axon (fiber) and the bottom portion (B) shows conduction over nonmylinated axon (fiber). These electrical signals will travel along the nerve fibers.

The information in the nervous system is coded by frequency of firing rather than the size of the action potential. This is shown schematically in FIG. 9. The bottom portion of the figure shows a train of action potentials.

In terms of electrical conduction, myelinated fibers conduct faster, are typically larger, have very low stimulation thresholds, and exhibit a particular strength-duration curve or respond to a specific pulse width versus amplitude for stimulation, compared to unmyelinated fibers. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Because of their very slow conduction, C fibers would not be highly responsive to rapid stimulation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well.

As shown in FIG. 10A, when the distal part of a nerve is electrically stimulated, a compound action potential is recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories as shown in the Table one below,

TABLE 1

| | Conduction | Fiber | |
|---|---|---|---|
| Fiber | Velocity | Diameter | |
| Type | (m/sec) | (μm) | Myelination |
| A Fibers | | | |
| Alpha | 70-120 | 12-20 | Yes |
| Beta | 40-70 | 5-12 | Yes |
| Gamma | 10-50 | 3-6 | Yes |
| Delta | 6-30 | 2-5 | Yes |
| B Fibers | 5-15 | <3 | Yes |
| C Fibers | 0.5-2.0 | 0.4-1.2 | No |

FIG. 10B further clarifies the differences in action potential conduction velocities between the Aδ-fibers and the C-fibers. For many of the application of current patent application, it is the slow conduction C-fibers that are stimulated by the pulse generator.

The modulation of nerve in the periphery, as done by the body, in response to different types of pain is illustrated schematically in FIGS. 11 and 12. As shown schematically in FIG. 11, the electrical impulses in response to acute pain sensations are transmitted to brain through peripheral nerve and the spinal cord. The first-order peripheral neurons at the point of injury transmit a signal along A-type nerve fibers to the dorsal horns of the spinal cord. Here the second-order neurons take over, transfer the signal to the other side of the spinal cord, and pass it through the spinothalamic tracts to thalamus of the brain. As shown in FIG. 12, duller and more persistent pain travel by another-slower route using unmyelinated C-fibers. This route made up from a chain of interconnected neurons, which run up the spinal cord to connect with the brainstem, the thalamus and finally the cerebral cortex. The autonomic nervous system also senses pain and transmits signals to the brain using a similar route to that for dull pain.

Vagus nerve stimulation, as performed by the system and method of the current patent application, is a means of directly affecting central function. FIG. 13 shows cranial nerves have both afferent pathway 19 (inward conducting nerve fibers which convey impulses toward the brain) and efferent pathway 21 (outward conducting nerve fibers which convey impulses to an effector). Vagus nerve is composed of 80% afferent sensory fibers carrying information to the brain from the head, neck, thorax, and abdomen. The sensory afferent cell bodies of the vagus reside in the nodose ganglion and relay information to the nucleus tractus solitarius (NTS).

The vagus nerve is composed of somatic and visceral afferents and efferents. Usually, nerve stimulation activates signals in both directions (bi-directionally). It is possible however, through the use of special electrodes and waveforms, to selectively stimulate a nerve in one direction only (unidirectionally). The vast majority of vagus nerve fibers are C fibers, and a majority are visceral afferents having cell bodies lying in masses or ganglia in the skull.

In considering the anatomy, the vagus nerve spans from the brain stem all the way to the splenic flexure of the colon. Not only is the vagus the parasympathetic nerve to the thoracic and abdominal viscera, it also the largest visceral sensory (afferent) nerve. Sensory fibers outnumber parasympathetic fibers four to one. In the medulla, the vagal fibers are connected to the nucleus of the tractus solitarius (viceral sensory), and three other nuclei. The central projections terminate largely in the nucleus of the solitary tract, which sends fibers to various regions of the brain (e.g., the thalamus, hypothalamus and amygdala).

As shown in FIG. 14, the vagus nerve emerges from the medulla of the brain stem dorsal to the olive as eight to ten rootlets. These rootlets converge into a flat cord that exits the skull through the jugular foramen. Exiting the Jugular foramen, the vagus nerve enlarges into a second swelling, the inferior ganglion.

In the neck, the vagus lies in a groove between the internal jugular vein and the internal carotid artery. It descends vertically within the carotid sheath, giving off branches to the pharynx, larynx, and constrictor muscles. From the root of the neck downward, the vagus nerve takes a different path on each side of the body to reach the cardiac, pulmonary, and esophageal plexus (consisting of both sympathetic and parasympathetic axons). From the esophageal plexus, right and left gastric nerves arise to supply the abdominal viscera as far caudal as the splenic flexure.

In the body, the vagus nerve regulates viscera, swallowing, speech, and taste. It has sensory, motor, and parasympathetic components. Table two below outlines the innervation and function of these components.

TABLE 2
Vagus Nerve Components

| Component | | |
|---|---|---|
| fibers | Structures innervated | Functions |
| SENSORY | Pharynx. larynx, | General sensation |
| | esophagus, external ear | |
| | Aortic bodies, aortic arch | Chemo- and baroreception |
| | Thoracic and abdominal | |
| | viscera | |
| MOTOR | Soft palate, pharynx, | Speech, swallowing |
| | larynx, upper esophagus | |
| PARA- | Thoracic and abdominal | Control of cardiovascular |
| SYMPATHETIC | viscera | system, respiratory and |
| | | gastrointestinal tracts |

On the Afferent side, visceral sensation is carried in the visceral sensory component of the vagus nerve. As shown in FIGS. 15A and 15B, visceral sensory fibers from plexus around the abdominal viscera converge and join with the right and left gastric nerves of the vagus. These nerves pass upward through the esophageal hiatus (opening) of the diaphragm to merge with the plexus of nerves around the esophagus. Sensory fibers from plexus around the heart and lungs also converge with the esophageal plexus and continue up through the thorax in the right and left vagus nerves. As shown in FIG. 15B, the central process of the nerve cell bodies in the inferior vagal ganglion enter the medulla and descend in the tractus solitarius to enter the caudal part of the nucleus of the tractus solitarius. From the nucleus, bilateral connections important in the reflex control of cardiovascular, respiratory, and gastrointestinal functions are made with several areas of the reticular formation and the hypothalamus.

The afferent fibers project primarily to the nucleus of the solitary tract (shown schematically in FIGS. 16 and 17) which extends throughout the length of the medulla oblongata. A small number of fibers pass directly to the spinal trigeminal nucleus and the reticular formation. As shown in FIG. 16, the nucleus of the solitary tract has widespread projections to cerebral cortex, basal forebrain, thalamus, hypothalamus, amygdala, hippocampus, dorsal raphe, and cerebellum. Because of the widespread projections of the Nucleus of the Solitary Tract, neuromodulation of the vagal afferent nerve fibers produce alleviation of symptoms of many of the neurological and neuropsychiatric disorders covered in this patent application.

### Background of Amplitude Modulation

There are two basic ways to produce amplitude modulation. The first is to multiply the carrier by a gain or attenuation factor that varies with the modulating signal. The second is to linearly mix or algebraically add the carrier and modulating signals, and then apply the composite signal to a non-linear device or circuit.

When the modulating signal is a sine wave (FIG. 18A) and carrier signal is sine wave (FIG. 18B), FIG. 18C shows an example of linearly mixed or algebraically added signal. The two signals have simply been added together or linearly mixed. Modulation is a multiplication process and not an addition process. When the composite waveform is applied to a non-linear device such as diode, the resulting waveform is shown in FIG. 18D. This resulting waveform across a tuned circuit is amplitude modulated and is shown in FIG. 18E.

FIG. 19 shows examples of amplitude modulation with complex modulation signal that can be produced for delivering to the vagus nerve, as some examples. The top part of FIG. 19 shows triangular wave modulation, the middle part of the figure shows rectangular wave modulation, and the bottom part shows a complex signal.

For the method and system of this application, virtually any shape signal from very simple to complex waveform shape can be generated and delivered to the vagus nerve via implanted stimulus receiver, as described later.

### PRIOR ART

One type of medical device therapy for neurological and neuropsychiatric disorders is generally directed to the use of an implantable lead and an implantable pulse generator technology or “cardiac pacemaker like” technology, i.e. stimulation with an implantable Neurocybernetic Prosthesis.

U.S. Pat. Nos. 4,702,254, 4,867,164 and 5,025,807 (Zabara) generally disclose animal research and experimentation related to epilepsy and the like and are directed to stimulating the vagus nerve by using “pacemaker-like” technology, such as an implantable pulse generator. The pacemaker technology concept consists of a stimulating lead connected to a pulse generator (containing the circuitry and DC power source) implanted subcutaneously or submuscularly, somewhere in the pectoral or axillary region, and programming with an external personal computer (PC) based programmer. Once the pulse generator is programmed for the patient, the fully functional circuitry and power source, are fully implanted within the patient's body. In such a system, when the battery is depleted, a surgical procedure is required to disconnect and replace the entire pulse generator (circuitry and power source). These patents neither anticipate practical problems of an inductively coupled system, nor suggest solutions to the same for an inductively coupled system for neuromodulation therapy.

U.S. Pat. No. 3,796,221 (Hagfors) is directed to controlling the amplitude, duration and frequency of electrical stimulation applied from an externally located transmitter to an implanted receiver by inductively coupling. Electrical circuitry is schematically illustrated for compensating for the variability in the amplitude of the electrical signal available to the receiver because of the shifting of the relative positions of the transmitter-receiver pair. By highlighting the difficulty of delivering consistent pulses, this patent points away from applications such as the current application, where consistent therapy needs to be continuously sustained over a prolonged period of time. The methodology disclosed is focused on circuitry within the receiver, which would not be sufficient when the transmitting coil and receiving coil assume significantly different orientation, which is likely in the current application.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) is directed to adjunct therapy of partial complex epilepsy and generalized epilepsy using an implanted lead-receiver and an external stimulator.

U.S. Pat. No. 5,807,397 (Barreras) is directed to an implantable stimulator with replenishable, high value capacitive power source.

U.S. Pat. No. 5,193,539 (Schulman, et al) is generally directed to an addressable, implantable microstimulator that is of size and shape which is capable of being implanted by expulsion through a hypodermic needle. In the Schulman patent, up to 256 microstimulators may be implanted within a muscle and they can be used to stimulate in any order as each one is addressable, thereby providing therapy for muscle paralysis.

U.S. Pat. No. 5,405,367 (Schulman, et al) is generally directed to the structure and method of manufacture of an implantable microstimulator.

The method and system of neuromodulation described in the current application, has several advantages over the prior art implantable pulse generator system. True modulation of the vagus nerve can be achieved by using a multilevel digital type of baseband signal, which is varied appropriately for the application and is software controlled. Further with this system and method, the therapy can be optimized, without regard to battery depletion as the power source is external, and surgical replacement of the pulse generator is avoided. The implanted hardware, can also be manufactured cheaper and with smaller size. Additionally, a new dimension of wireless communication and control of pulse generator is more practical.

### SUMMARY OF THE INVENTION

The system and method of the current invention also overcomes many of the disadvantages of the prior art by simplifying the implant and taking the programmability into the external stimulator. Further, the programmability of the external stimulator can be controlled remotely, via the wireless medium, as described in a co-pending application. The system and method of this invention uses the patient as his/her own feedback loop. Once the therapy is prescribed by the physician, the patient can receive the therapy as needed based on symptoms, and the patient can adjust the stimulation within prescribed limits for his/her own comfort.

Accordingly in one aspect of the invention, modulated pulses are mixed with carrier signal, and these modulated high frequency pulses are used to modulate the vagus nerve of a patient, to deliver therapy.

In another aspect of the invention, the modulation and stimulation parameters can be adjusted for optimizing therapy for different neurological disorders.

In another aspect of the invention, the modulation and stimulation parameters can be adjusted for optimizing therapy for the individual patient.

In another aspect of the invention, the external pulse generator is inductively coupled to an implanted stimulus receiver, which does not contain an internal power supply.

In another aspect of the invention, the external pulse generator is inductively coupled to an implanted stimulus receiver, which has an implanted power source.

In another aspect of the invention, the external pulse generator contains a telemetry module, whereby therapy can be controlled remotely.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram of the structure of a nerve.

FIG. 2 is a diagram showing different types of nerve fibers.

FIGS. 3A and 3B are schematic illustrations of the biochemical makeup of nerve cell membrane.

FIG. 4 is a figure demonstrating subthreshold and suprathreshold stimuli.

FIGS. 5A, 5B, 5C are schematic illustrations of the electrical properties of nerve cell membrane.

FIG. 6 is a schematic illustration of electrical circuit model of nerve cell membrane.

FIG. 7 is an illustration of propagation of action potential in nerve cell membrane.

FIG. 8 is an illustration showing propagation of action potential along a myelinated axon and non-myelinated axon.

FIG. 9 is an illustration showing a train of action potentials.

FIG. 10A is a diagram showing recordings of compound action potentials.

FIG. 10B is a schematic diagram showing conduction of first pain and second pain.

FIG. 11 is a schematic illustration showing mild stimulation being carried over the large diameter A-fibers.

FIG. 12 is a schematic illustration showing painful stimulation being carried over small diameter C-fibers

FIG. 13 is a schematic diagram of brain showing afferent and efferent pathways.

FIG. 14 is a schematic diagram showing the vagus nerve at the level of the nucleus of the solitary tract.

FIG. 15A is a schematic diagram showing the thoracic and visceral innervations of the vagal nerves.

FIG. 15B is a schematic diagram of the medullary section of the brain.

FIG. 16 is a block diagram illustrating the connections of solitary tract nucleus to other centers of the brain.

FIG. 17 is a schematic diagram of brain showing the relationship of the solitary tract nucleus to other centers of the brain.

FIGS. 18A, 18B, 18C, 18D, 18E, are diagrams illustrating amplitude modulation.

FIG. 19 is a diagram illustrating waveforms capable with amplitude modulation.

FIG. 20 is a block diagram for delivering amplitude modulated electrical pulses to an implanted subcutaneous coil.

FIG. 21 is an electrical circuit diagram of a Colpitt's oscillator.

FIG. 22A, 22B, and 22C is a schematic diagram showing an integrated-circuit (IC) waveform generator.

FIG. 23 is a block diagram of a modulator.

FIGS. 24A, 24B, 24C, 24D, 24E, 24F, 24G, 24H are diagrams of amplitude modulated waveforms for modulating the vagus nerve.

FIG. 25 is a block diagram of the external pulse generator.

FIG. 26 is a schematic diagram showing an implanted stimulus receiver in electrical contact with the vagus nerve.

FIGS. 27A, 27B, and 27C are schematic diagrams showing circuitry for implanted stimulus receivers.

FIG. 28 is a schematic diagram showing the implantable lead stimulus-receiver.

FIG. 29 is a schematic diagram showing customized garment with a pocket for the placement of the external (primary) coil of the transmitter.

FIG. 30 is a block diagram showing schematically the functioning of the external transmitter and the implanted lead stimulus-receiver.

FIG. 31 is a schematic diagram showing a workable Class-D driver.

FIGS. 32A and 32B are electrical diagrams showing the concept of Class-E amplifier.

FIG. 33 is a schematic diagram showing a workable Class-E driver.

FIG. 34 is a schematic block diagram showing a system for neuromodulation of the vagus nerve with an implanted component which is both RF coupled and contains a battery.

FIG. 35 is a schematic diagram showing wireless communication with the stimulus generator and a remote computer.

FIG. 36 is a schematic block diagram showing communication of stimulus generator over the wireless internet.

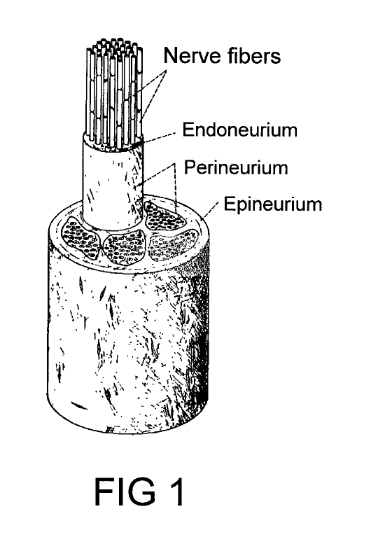

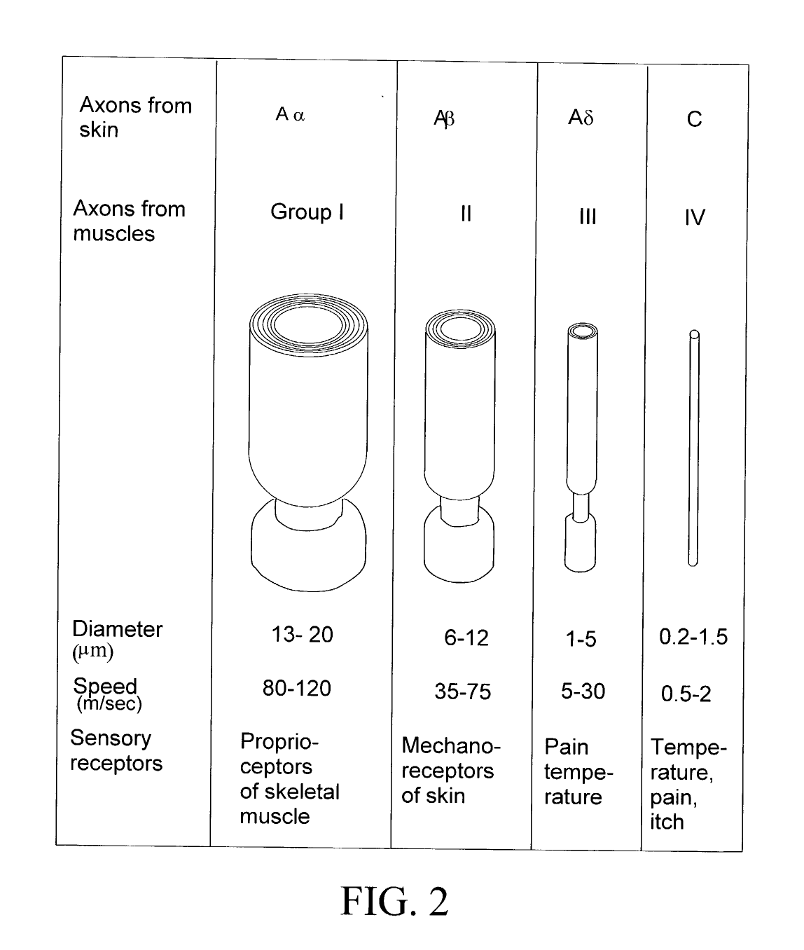

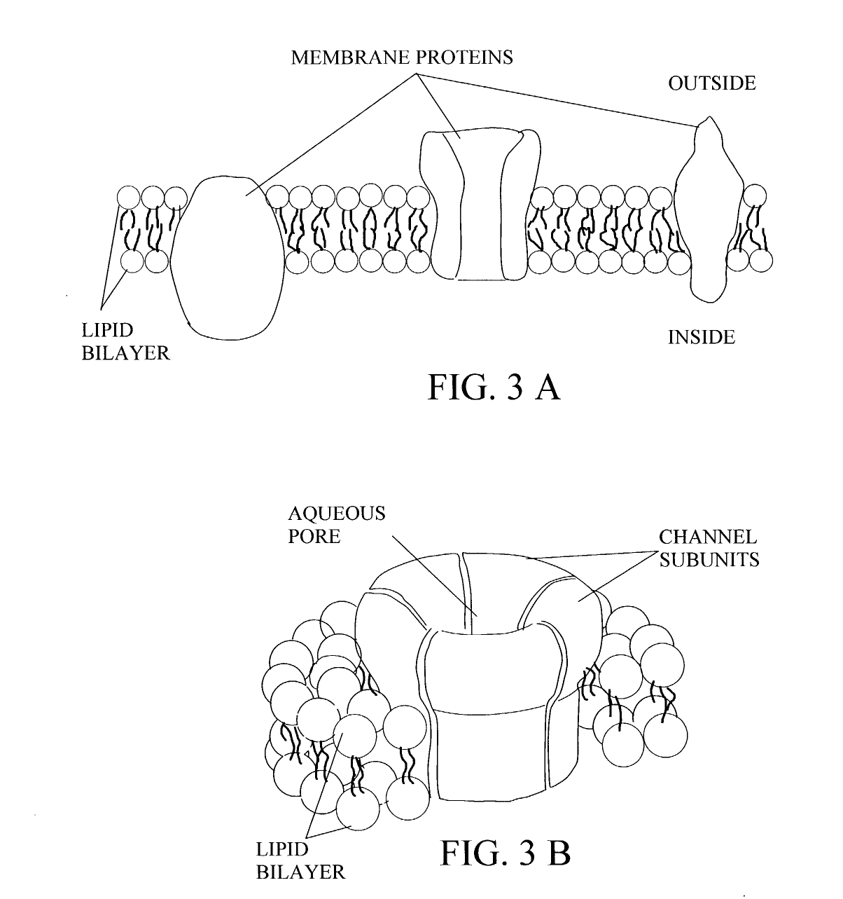

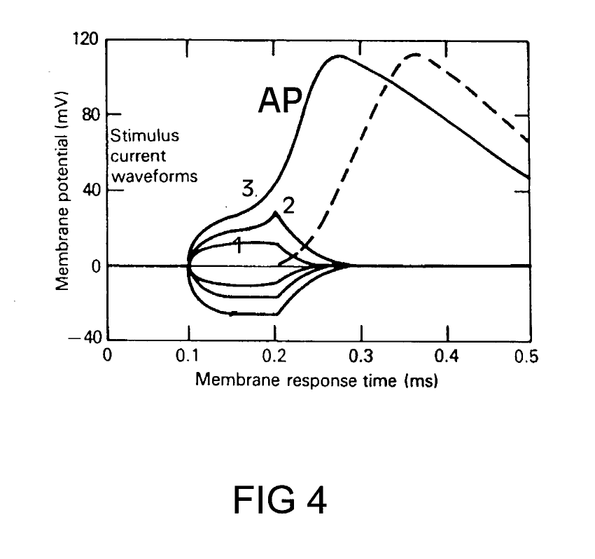

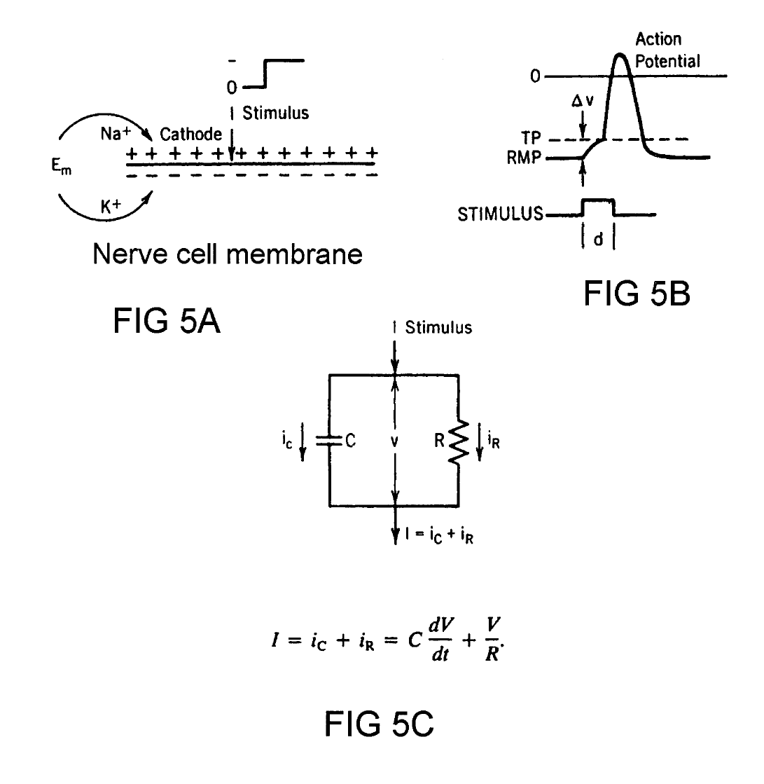

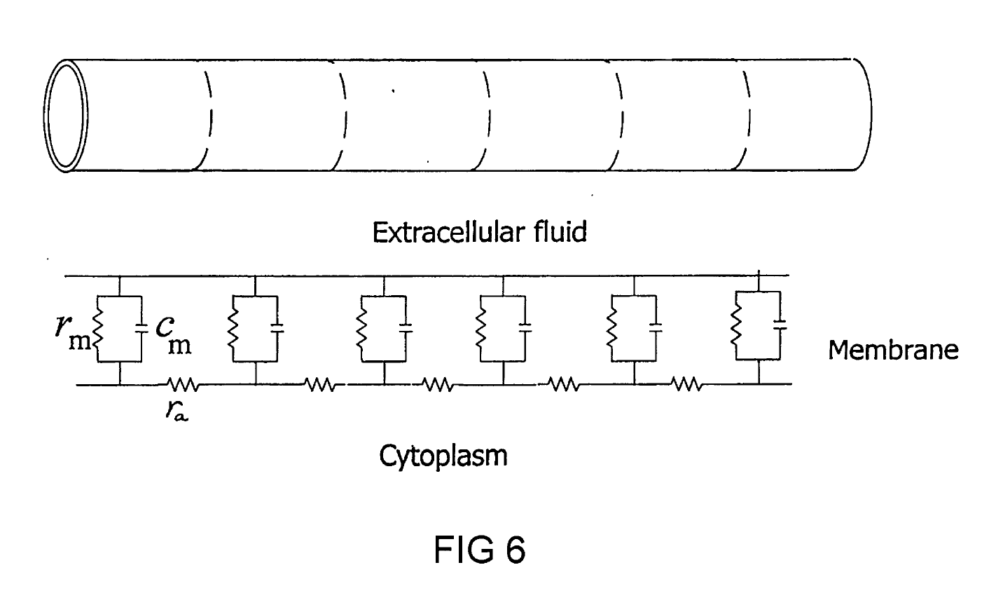

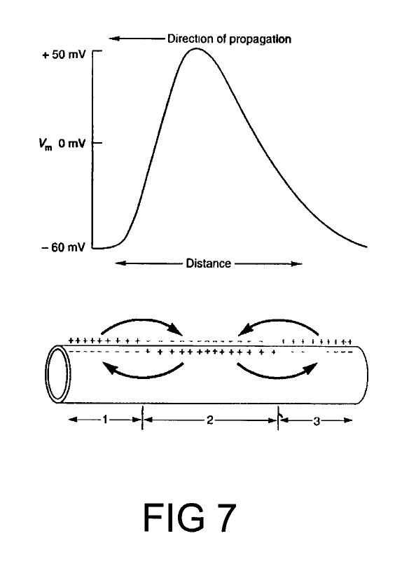

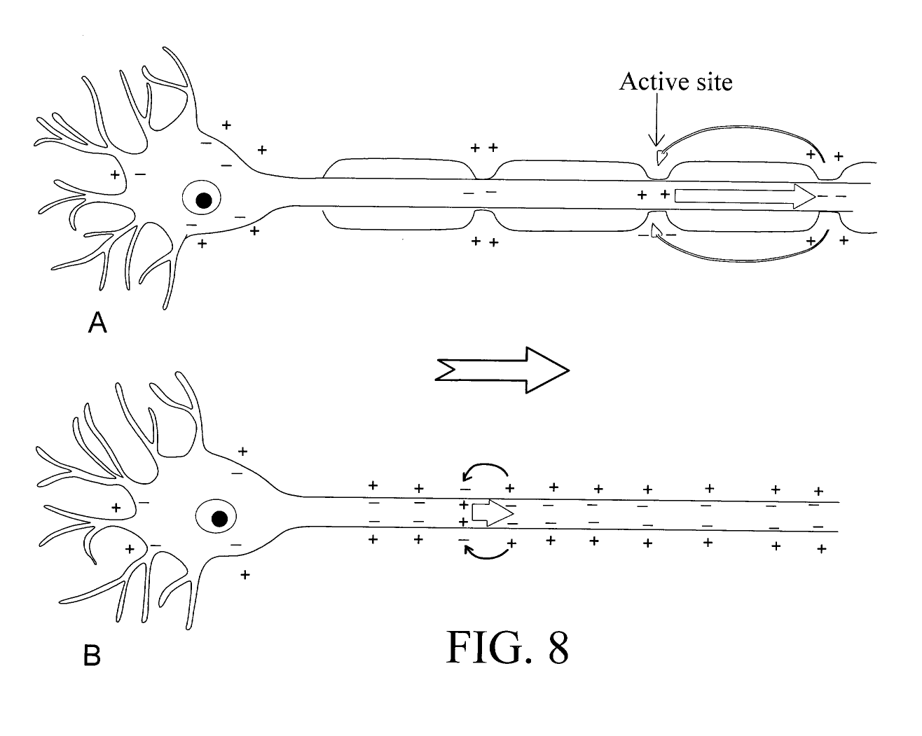

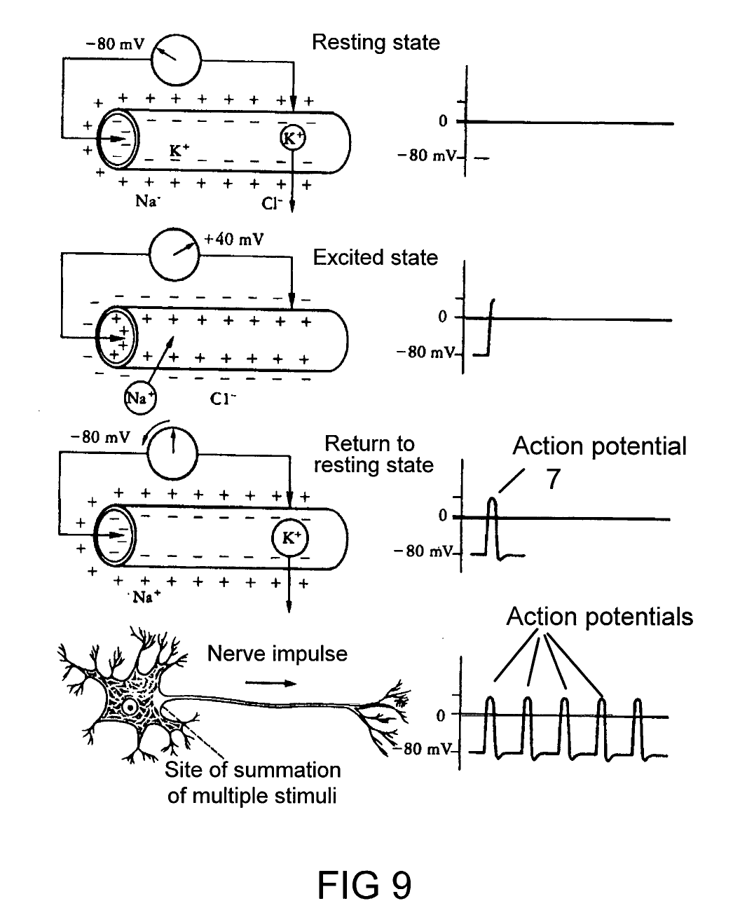

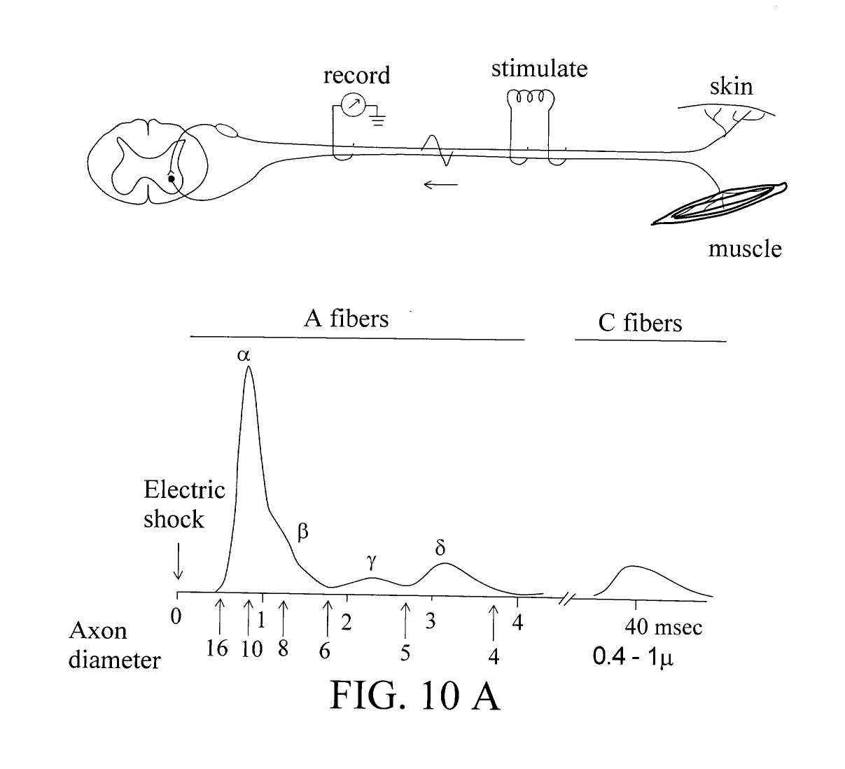

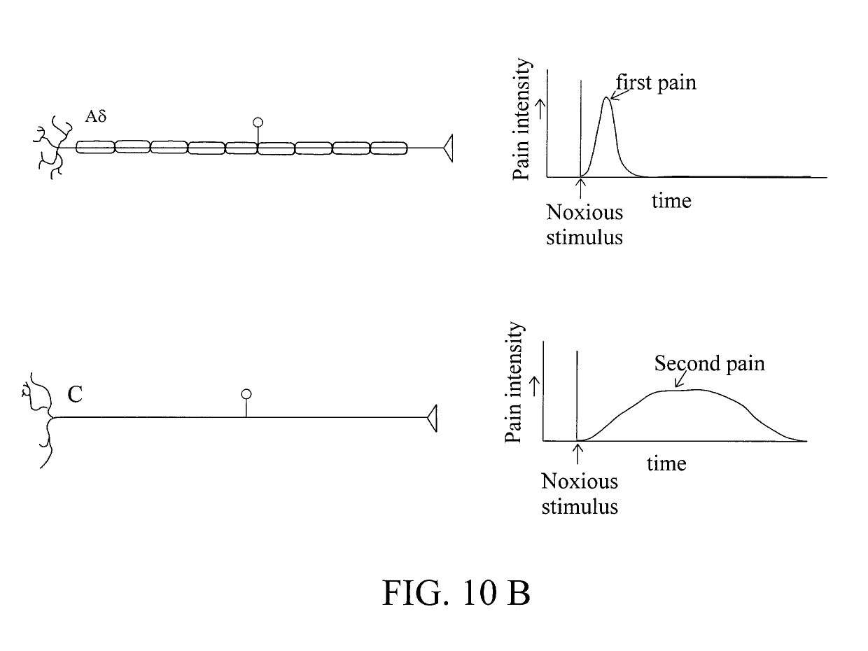

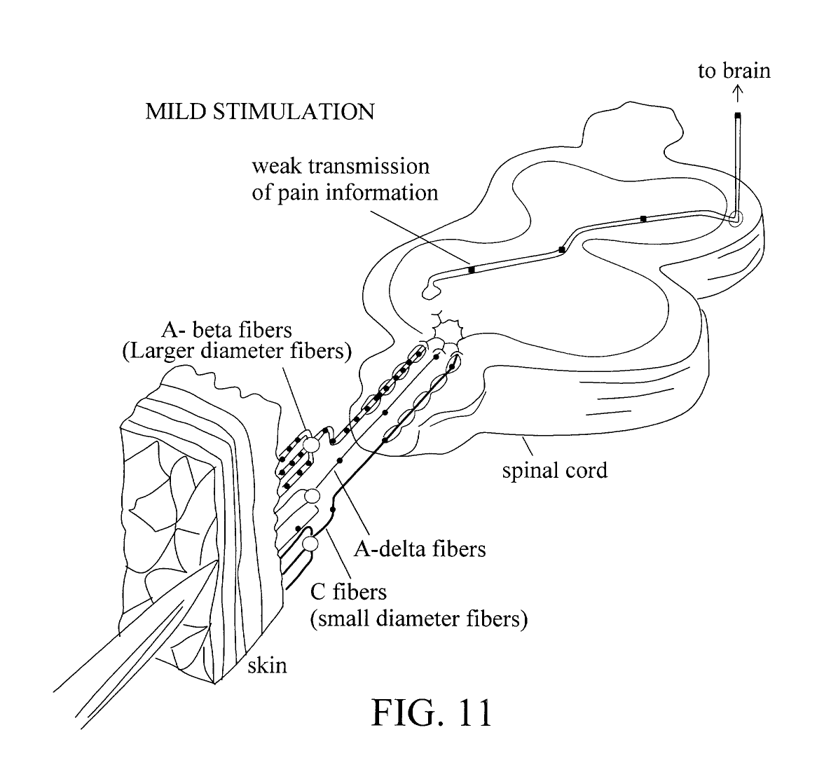

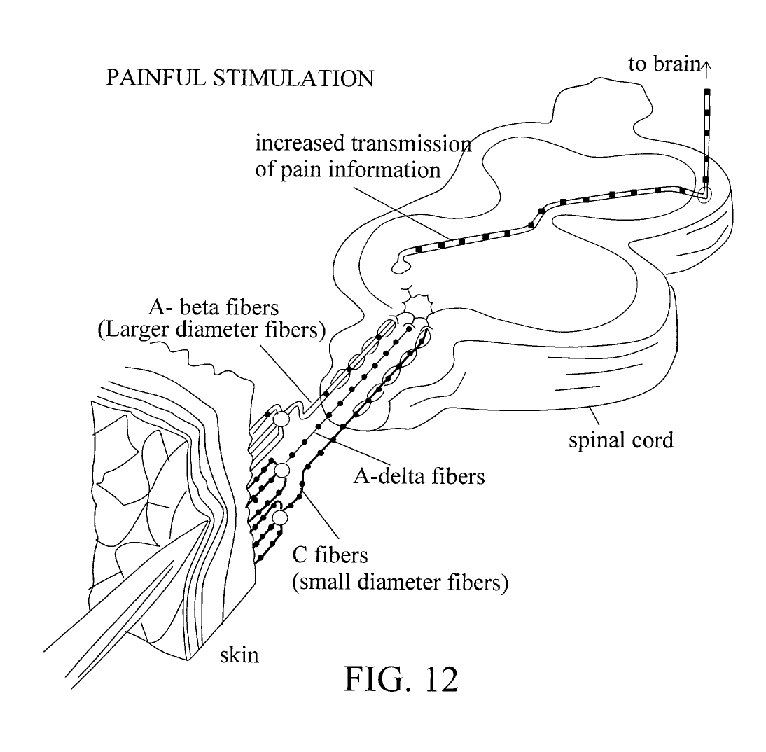

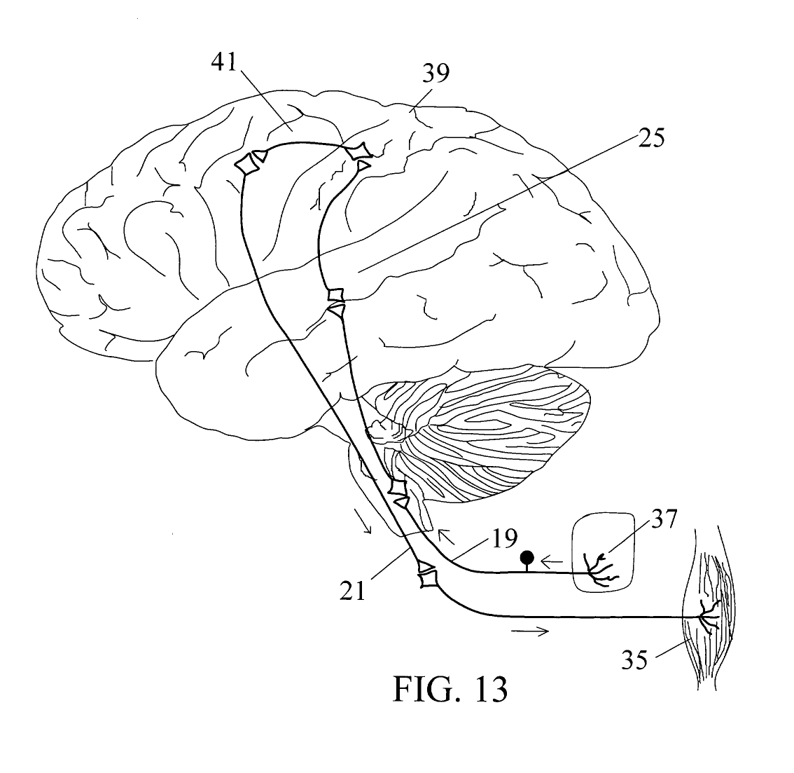

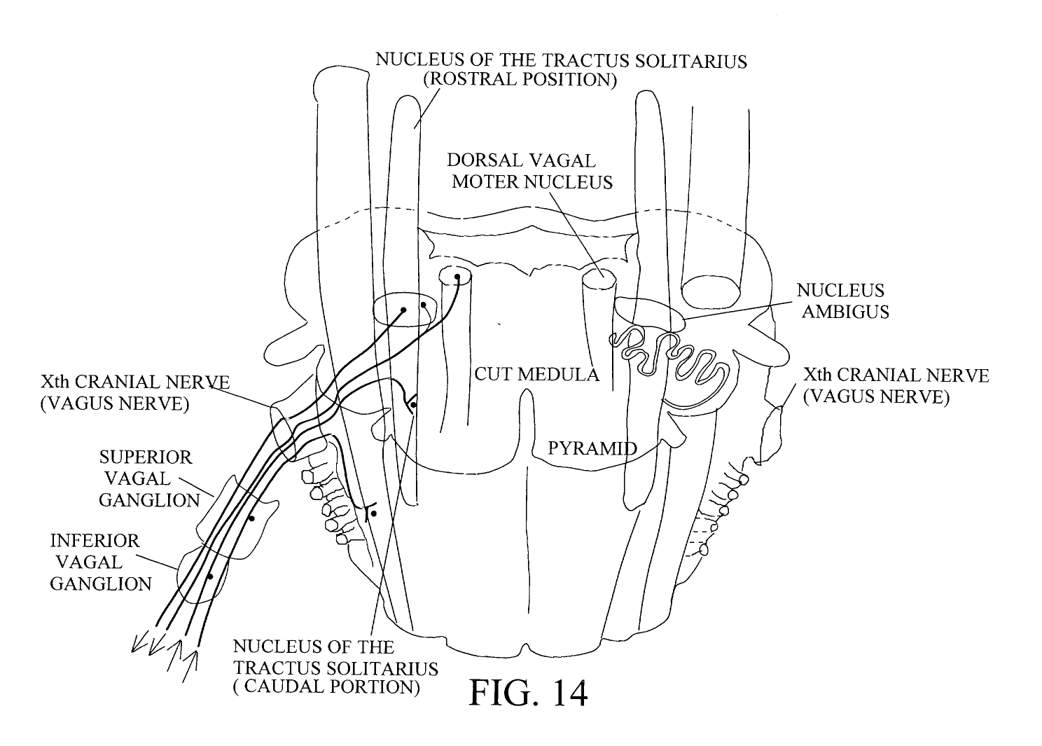

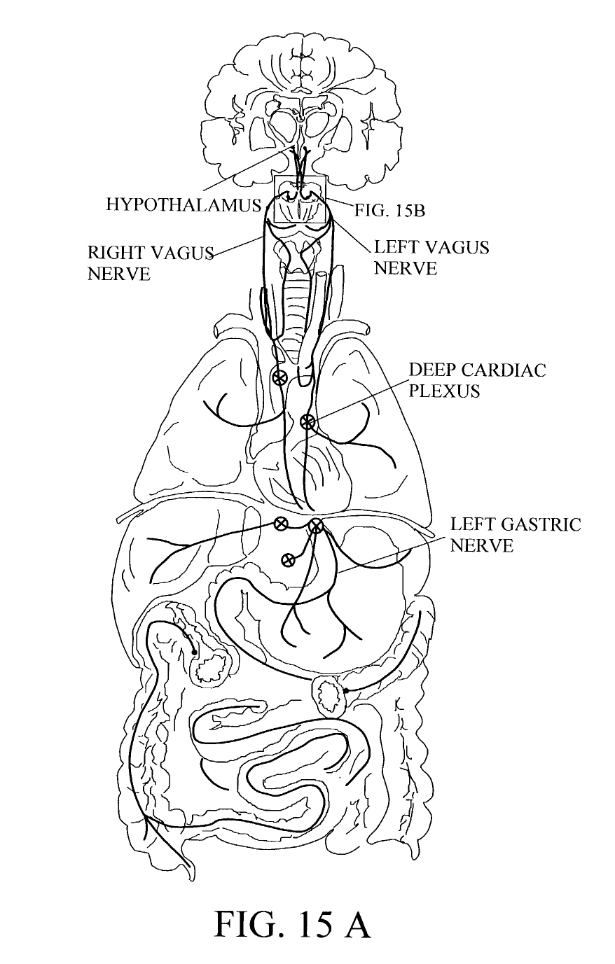

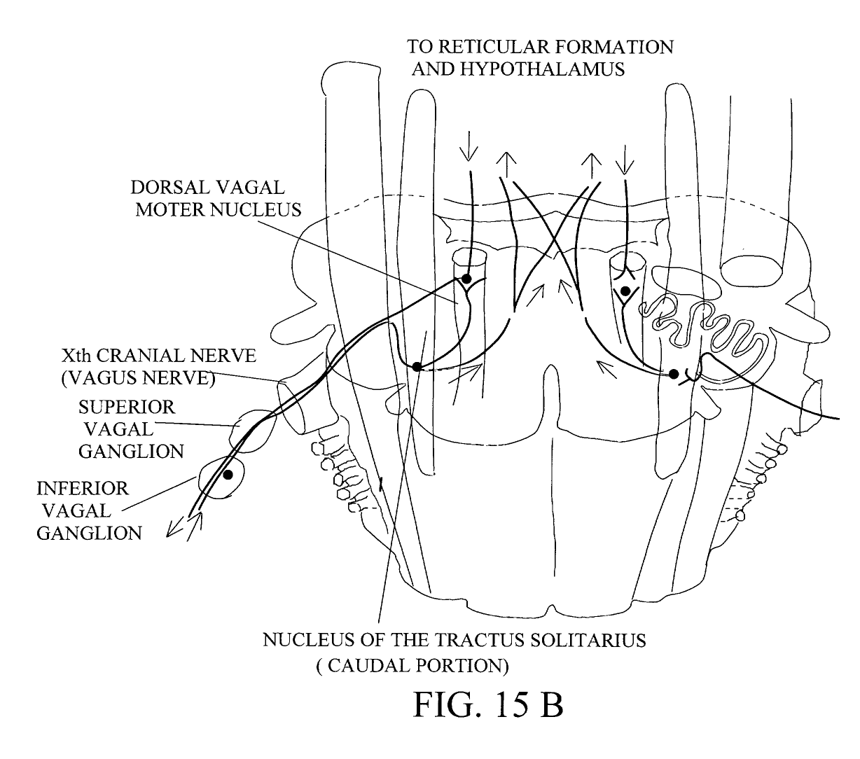

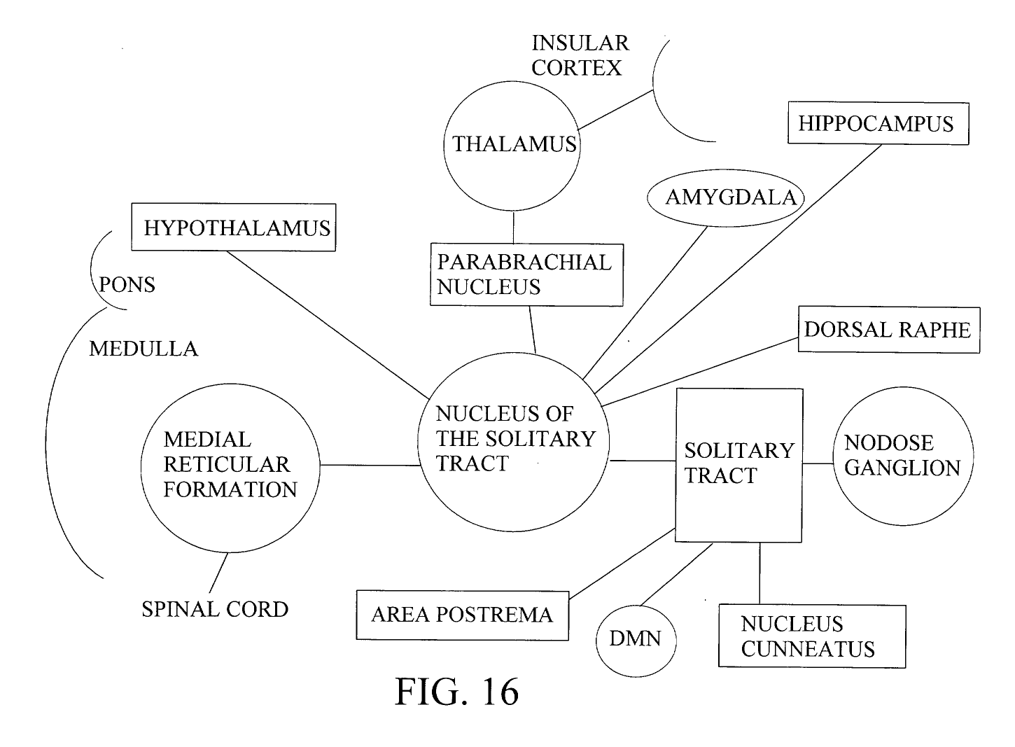

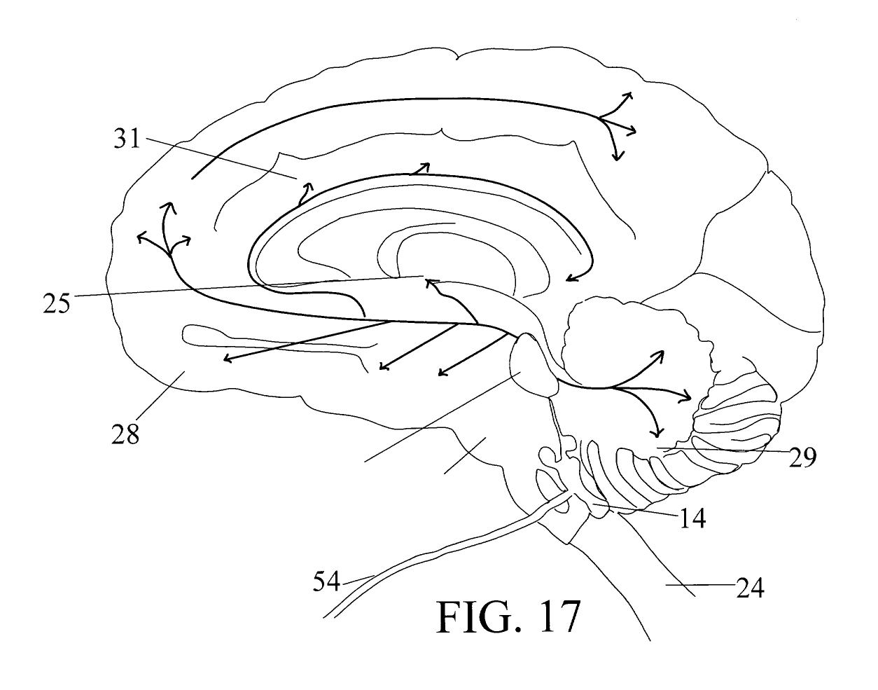

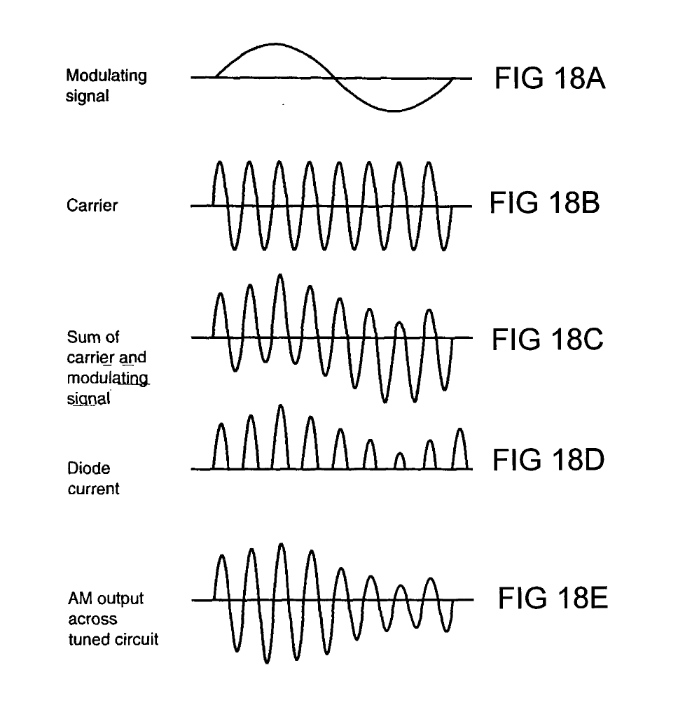

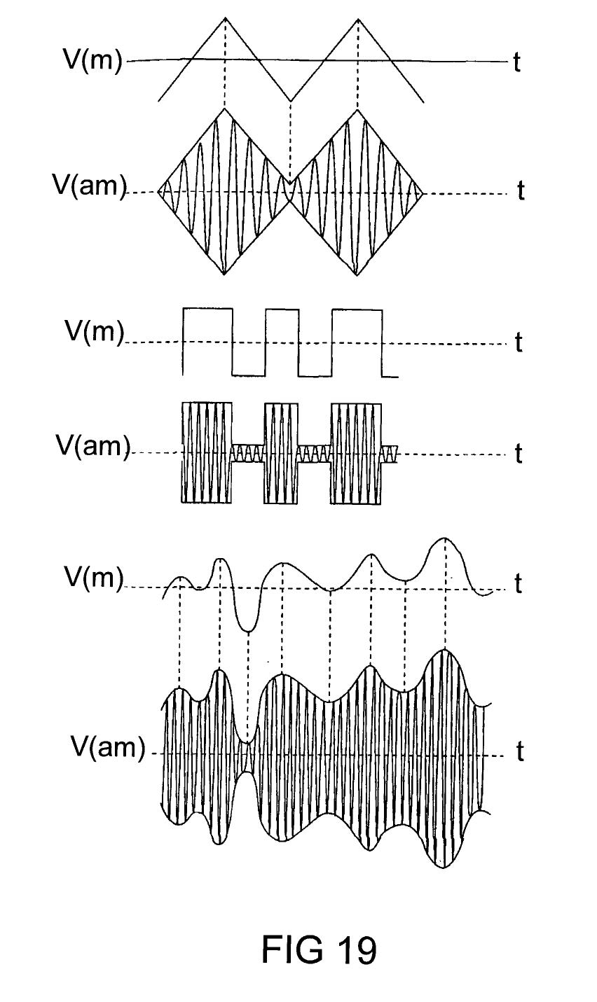

### DETAILED DESCRIPTION OF THE INVENTION

The following description is of the current embodiment for carrying out the invention. This description is not to be taken in a limiting sense, but is made merely for the purpose of describing the general principles of the invention. The scope of the invention should be determined with reference to the claims.

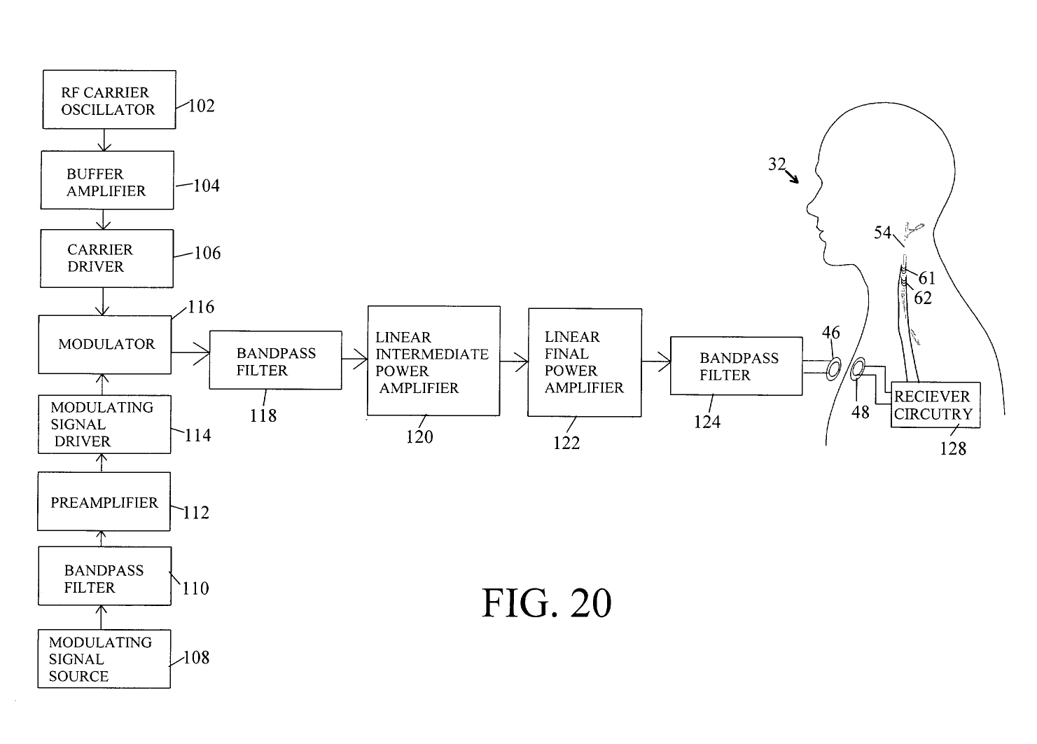

The selective stimulation of various nerve fibers of a cranial nerve such as the vagus nerve (or neuromodulation of the vagus nerve), as performed by the system and method of this invention is shown schematically in FIG. 20, as a block diagram. A modulator 116 receives analog (sine wave) high frequency “carrier” signal and modulating signal. The modulating signal can be multilevel digital, binary, or even an analog signal. In the presently preferred embodiment, mostly multilevel digital type modulating signals are used. The modulated signal is amplified 120,122, conditioned 124, and transmitted via a primary coil 46 which is external to the body. A secondary coil 48 of an implanted stimulus receiver, receives, demodulates, and delivers these pulses to the vagus nerve 54 via electrodes 61 and 62. The receiver circuitry 128 is described later.

The carrier frequency is optimized. Presently preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

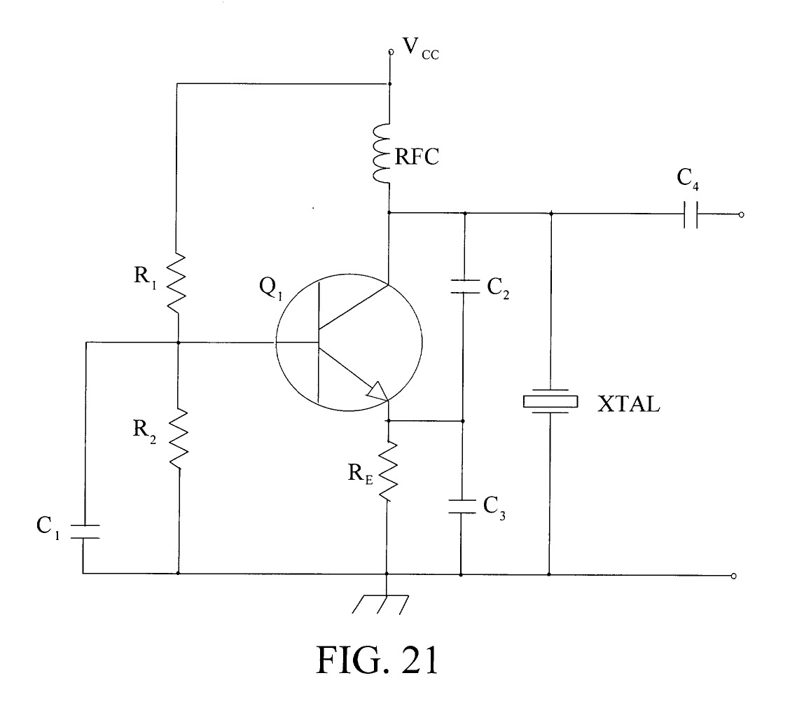

For generating carrier frequency, standard crystal controlled oscillator such as Colpitts or Hartley's oscillator may be used. FIG. 21 shows an example of Colpitts crystal oscillator. As shown in the figure, AC feedback circuit consists of the capacative voltage divider, C₂ and C₃, and the crystal element. A portion of the AC signal developed at the collector of Q1 provides regenerative feedback to the emitter. The amplitude of this AC feedback signal is determined by the ratio of C₂ and C₃. The resonant-frequency characteristics of the crystal controls the frequency of the AC signal developed at the collector. The output signal is taken from the collector terminal via DC blocking capacitor C4. Resistors R₁, R₂, and R_{E} establish the operating bias for the base of Q1. Although a bipolar NPN transistor is shown here, FETs can be used in the Colpitts crystal oscillator with appropriate circuit changes. As shown in blocks 104 and 106 of FIG. 20, these high frequency “carrier” signals are amplified before they are modulated.

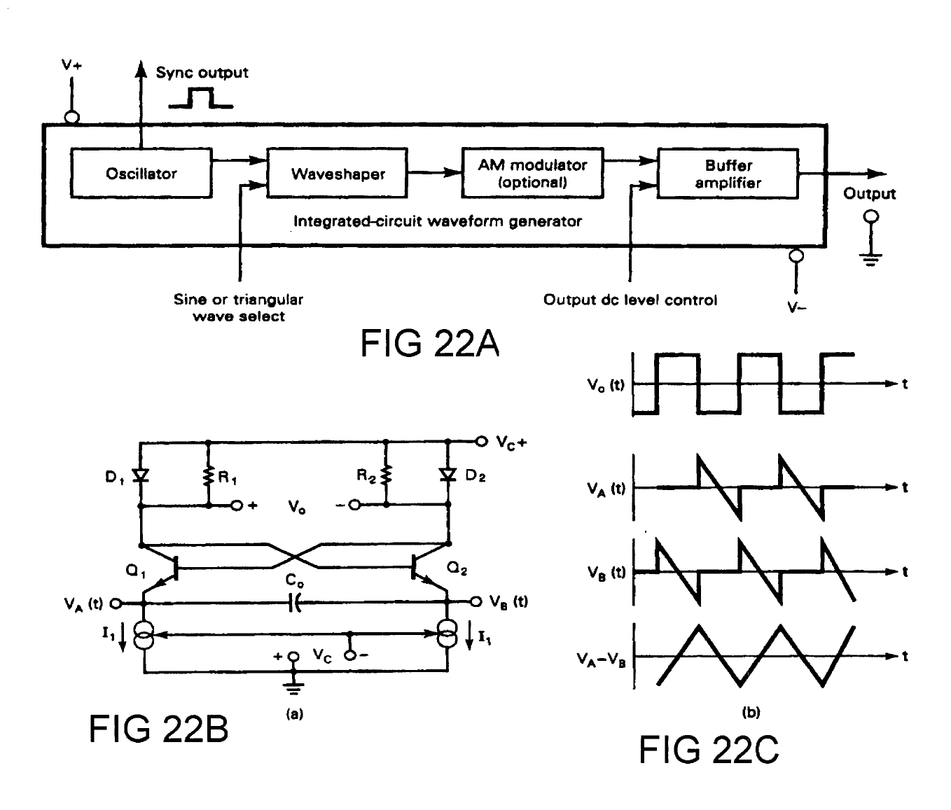

An example of modulating signal source is shown in FIG. 22A as an integrated-circuit (IC) waveform circuit. The oscillator section generates the basic oscillator frequency, and the waveshaper circuit converts the output from the oscillator to either a sine-, square-, triangular-, or ramp-shaped waveform (FIG. 22C). The modulator, when used, allows the circuit to produce amplitude-modulated signals, and the output buffer amplifier isolates the oscillator from its load and provides a convenient place to add DC levels to the output waveform. The sync output can be used either as a square-wave source or as a synchronizing pulse for external timing circuitry. A typical IC oscillator circuit utilizes the constant-current charging the discharging of external timing capacitors. FIG. 22B shows the simplified schematic diagram for such a waveform generator that uses an emitter-coupled multivibrator, which is capable of generating square waves as well as triangle and linear ramp waveforms. The circuit operates as follows. When transistor Q₁ and D1 are conducting, transistor Q₂ and diode D₂ are off, and vice versa. This action alternately charges and discharges capacitor Cₒ from constant current source 11. The voltage across D₁ and D₂ is symmetrical square wave with a peak-to -peak amplitude of 2 V_{BE}. V_{A} is constant when Q₁ is on but becomes a linear ramp with a slope equal to −I₁/C₀ when Q₁ goes off. Output V_{B}(t) is identical to V_{A}/t₀, except it is delayed by a half-cycle. Differential output, V_{A}(t)−V_{B}(t), is a triangle wave. FIG. 22C shows the output voltage waveforms typically available.

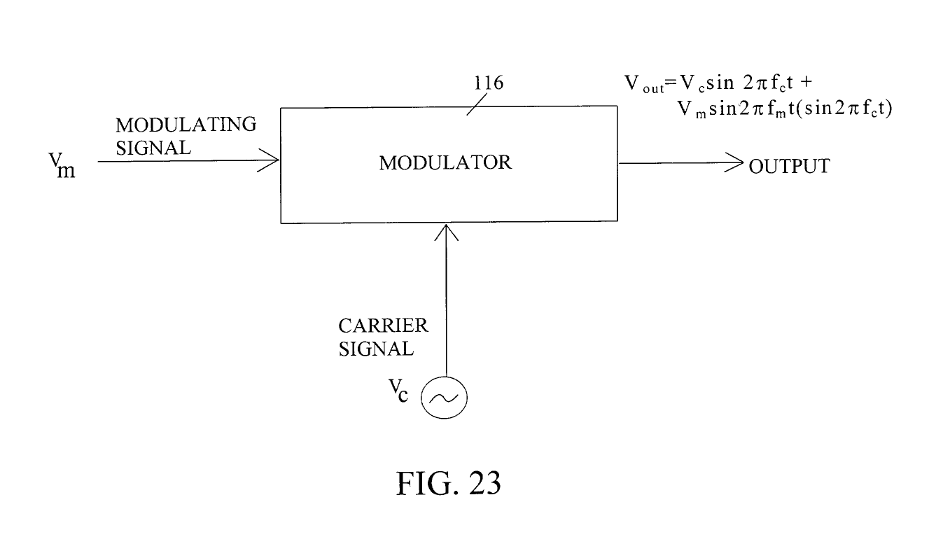

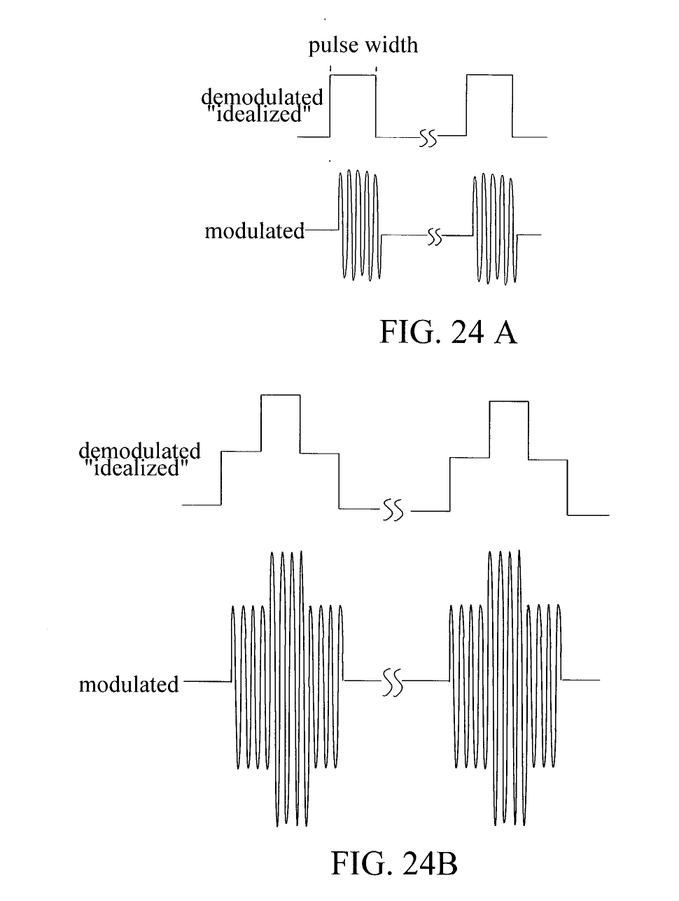

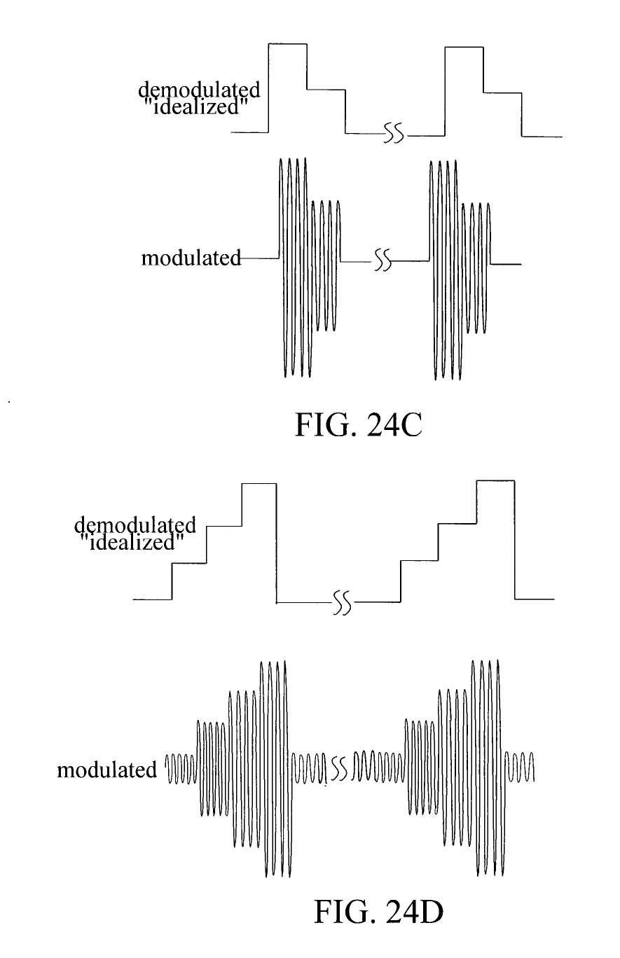

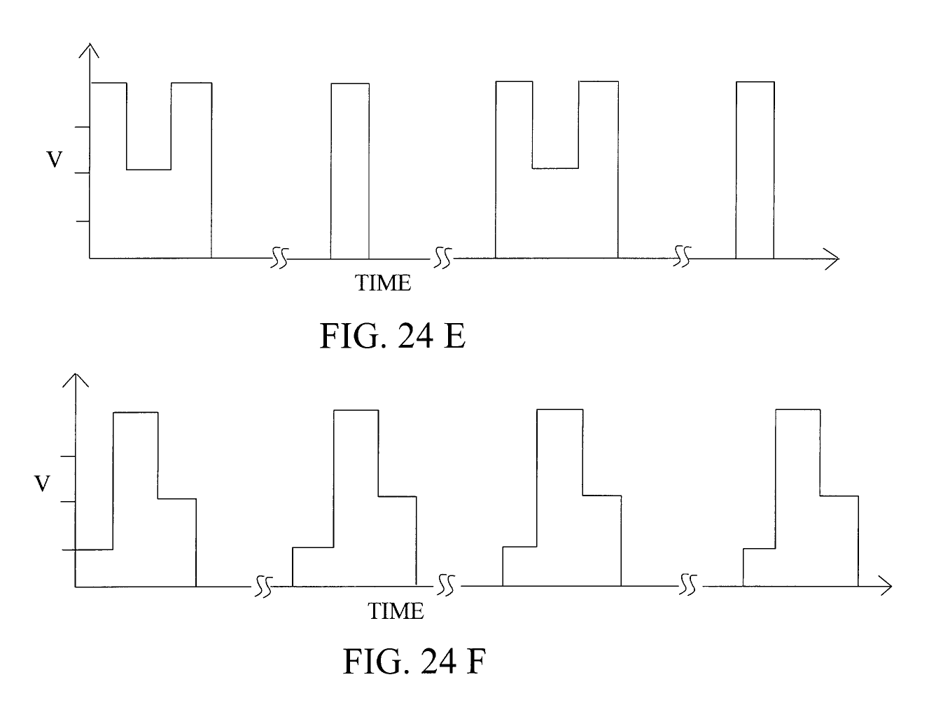

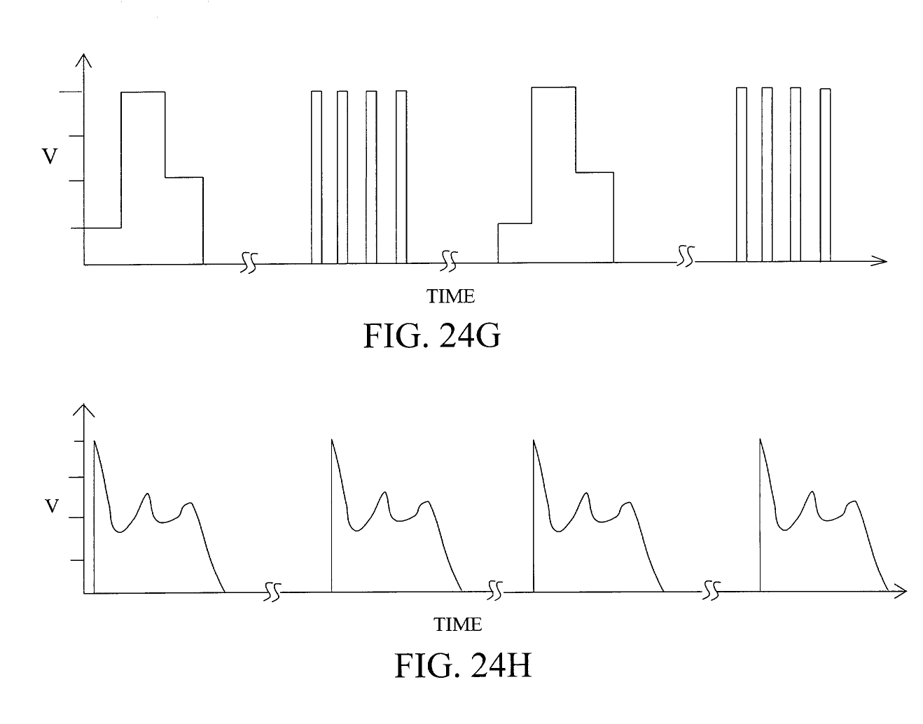

The analog carrier signals are modulated at the modulator 116, as shown in FIG. 23. The modulating signals may be digital or analog. In the presently preferred embodiment, mostly multilevel digital signals are being employed. The modulation needs to be through a non-linear device. As shown by the formula in FIG. 23, non-linear modulation which is achieved using a non-linear device such as a diode, transistor, or an IC is employed at the output. These modulated high frequency pulses are amplified 120, filtered 122,124 and transmitted via an external primary coil 46 as shown in the FIG. 20. FIGS. 24A-24H show examples of the complex waveform that are able to be achieved with non-linear mixing of multi-level digital signal with a constant frequency carrier signal. In FIGS. 24A-24D, modulated signals are shown in the bottom part of the figure and the demodulated signals are shown in top part of the figure. In FIGS. 24E to 24H only the “idealized” demodulated signals are shown. Further, the modulating signal can be constantly changing in a certain pattern, which is selectable and programmable in the pulse generator, as described later.

The method and system described here, is very versatile for delivering virtually any form, any sequence, and any time intervals called for in the application. As medical knowledge advances, different modulation schemes that are elucidated by clinical research can be programmed in the baseband signal, for delivering therapy for various neurological disorders, such as epilepsy, depression, anxiety, Alzheimer's disease and the like. Neuromodulation for different applications is described later.

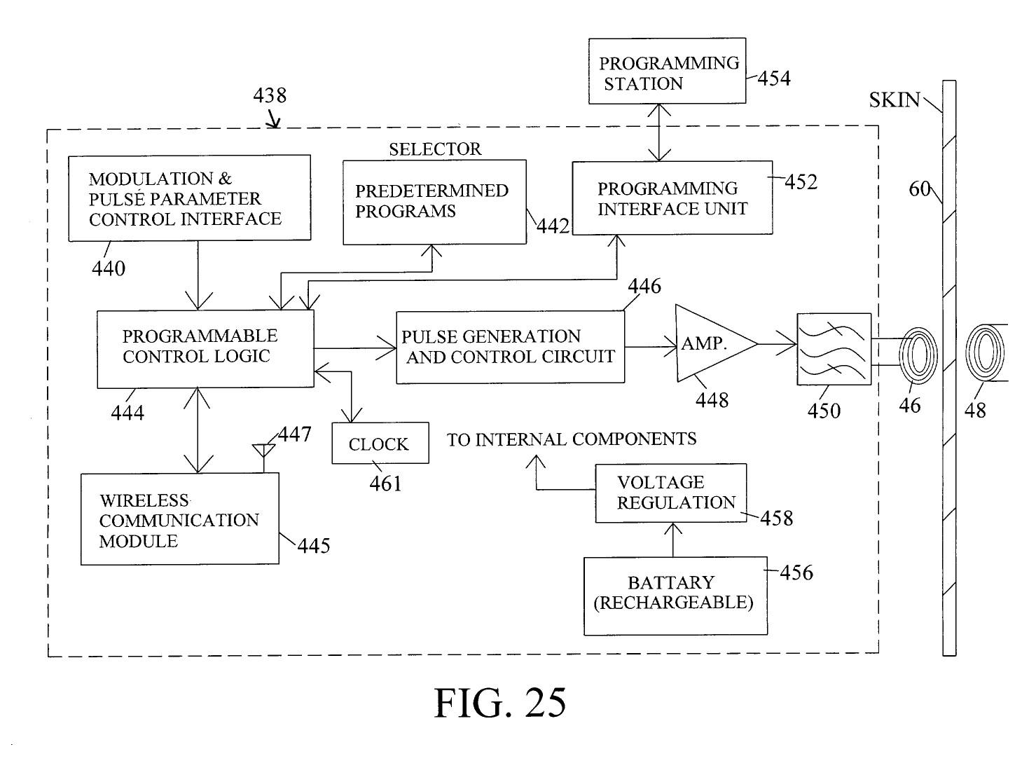

FIG. 25 shows a simplified block diagram of the external pulse generator used in this embodiment. Programmable control logic 444 having inputs from pre-determined programs selector 442 and pulse parameter control interface 440. The programmable control logic 444 controls the pulse generation circuitry 446. The electrical signals once generated are amplified 448 band-pass filtered 450 and transmitted through the primary coil (antenna) 46. The control logic 444 of the pulse generator using internal clock 461 having a crystal oscillator to provide timing signals for device operation. The programmable control logic 444 can also be interfaced to a programming station 454 via a standard type of communication interface such as RS232-C serial interface. New pre-determined programs may be loaded into the external pulse generator system 438. A battery 456, along with voltage regulator 458 supplies power to internal components.

In one embodiment, the external pulse generator 438 also contains a wireless communication module 445 for remote control and remote activation of pre-determined programs that may be locked-out to the patient. The methodology for that is described in a co-pending application Ser. No. 09/837565, and is incorporated here by reference.

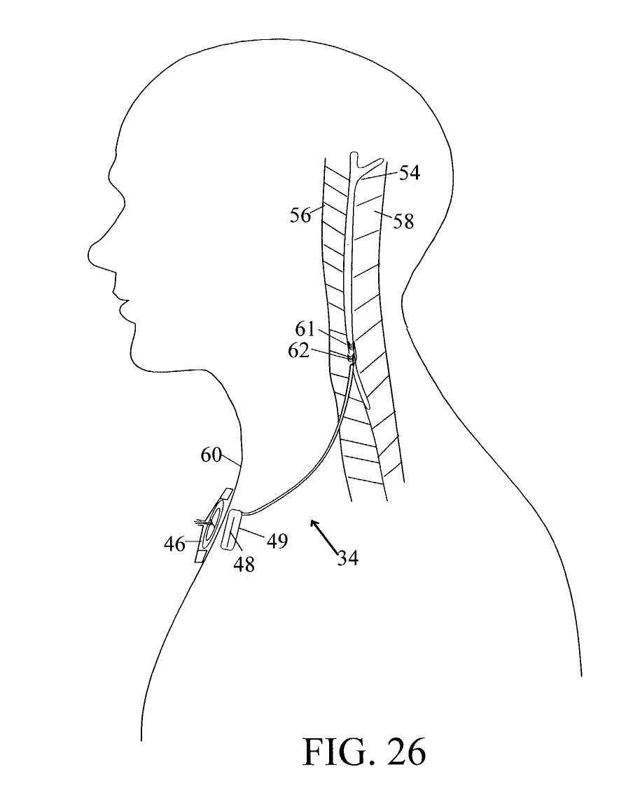

The electrical pulses transmitted via the primary (external) coil 46 are inductively coupled to an implanted secondary coil 48, which is in electrical connection with the vagus nerve 54, as shown in FIG. 26. The circuitry within the implanted lead-receiver may be completely passive (RF coupled), or may combine RF coupling and a battery powered system.

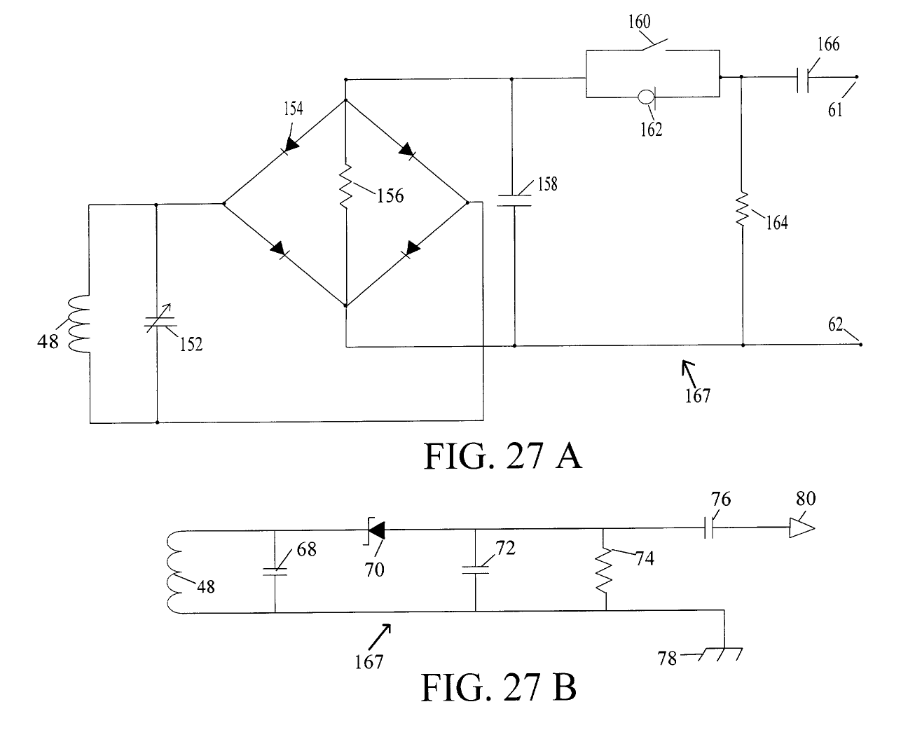

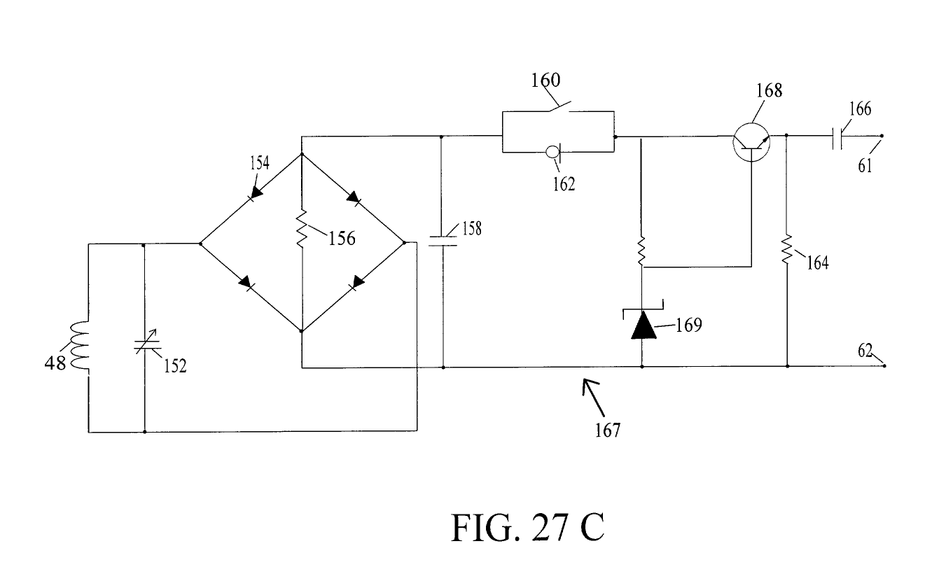

The circuitry contained in the proximal end 49 of the passive implantable stimulus receiver 34 is shown schematically in FIGS. 27A, 27B, 27C. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In the current embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, used in the current embodiment. Unipolar stimulation is more likely to stimulate skeletal muscle in addition to nerve stimulation. The implanted circuit 167 for this embodiment is passive, so a battery does not have to be implanted.

The circuitry shown in FIGS. 27B and 27C can be used as an alternative, for the implanted stimulus receiver. The circuitry of FIG. 27B is a slightly simpler version, and circuitry of FIG. 27C contains a conventional NPN transistor 168 connected in an emitter-follower configuration. In an alternative embodiment, the implanted stimulus receiver may be RF coupled and a battery powered system.

As mentioned earlier, this system and method of neuromodulation is very versatile, for delivering adjunct therapy for various neurologic and neuropsychiatric disorders. The modulation or baseband signal can be software controlled. As further medical insight is gained with afferent vagal stimulation, new waveform morphology and stimulation program can be re-loaded into the pulse generator. U.S. Pat. No. 6,366,814, describes an external pulse generator where new programs can be re-loaded, and is incorporated here by reference.

Afferent neuromodulation of the vagus nerve has beneficial therapy effects for various neurological, neuropsychiatric, and psychiatric disorders such as various forms of epilepsy, depression, anxiety disorders, compulsive obsessive disorders, eating disorders, obesity and dementia including Alzheimer's disease among others.

Different levels of neuromodulation are required for optimal therapy of the above different disorders. For example, adjunct therapy of partial complex epilepsy and generalized epilepsy appears to be dependent upon C-fibers carrying nerve impulses to Nucleus of the Solitary tract in the Medullary centers of the brain. Further, therapy benefits also appear to have cumulative effect over time, i.e. 1-year of stimulation therapy appears to be more effective than 1 to 2 months of stimulation therapy. For adjunct treatment of anxiety disorders and depression, the relative contribution of C-fibers appears to be less important. The best neuromodulation schemes for Alzheimer's disease are not completely understood, and may turn out to be quite complex.

In one clinical study, A-beta fibers responded very well to high frequency stimulation, e.g. 100 Hz with an intensity just above threshold. In another study, A-beta fibers appeared to respond also to low-frequency stimulation (2 Hz) with an intensity which triggered visible muscular twitches. Activation of A-delta and C-fibers is usually caused by low-frequency stimulation (less than 10 Hz) with an intensity well above threshold. To activate all three types of afferent nerve fibers, high-frequency and low-frequency stimulation has to be combined in one treatment.

In a paper published by Scherder et al, in Behavioral Brain Research 67 (1995) 211-219, the authors showed that using transcutaneous electrical nerve stimulation (TENS), beneficial effects were achieved in patients with early stage of Alzheimers' disease using asymmetric biphasic square pulses, applied in bursts of trains, nine pulses per train, with an interval frequency of 160 Hz, a repetition rate of 2 Hz, and a pulse width of 40 μs.

The optimal afferent neuromodulation patterns for therapy of complex disorders such as Alzheimer's disease and depression are evolving, as more understanding of the mechanisms are gained. The system and method of this invention is suited for adaptation to “state of art” electrical stimulation pulse patterns as they emerge, based on clinical research and clinical understanding.

Simple pulses such as shown in FIG. 24A are suited for stimulating C fibers (and A & B) fibers, if delivered to the nerve electrode for 200-500 μS pulse widths, 1-4 mAmp amplitude, and stimulation frequency around 20-50 HZ. A complex pulse waveform such as shown in FIG. 24F can be divided into 3 phases. Phase 1 will tend to stimulate A & B fibers, the middle portion of the pulse, which is larger amplitude will tend to recruit C-fibers as will, and the third phase of the pulse will be effective for stimulating only the larger diameter or mylinated fibers, such as the A fibers. Additionally, the high frequency delivery pulses can be constantly changing to change the neuromodulation of the vagus nerve to adapt to an individual patient or a specific disease state.

The “tuning” of the vagus nerve or another cranial nerve can be performed in one of two ways. One method is to activate one of several “pre-determined” programs. A second method is to “custom” program the electrical parameters which can be selectively programmed, for specific disease state of the individual patient. The electrical parameters which can be individually programmed, include variables such as pulse amplitude, pulse width, frequency of stimulation, modulation type, modulation index, stimulation on-time, and stimulation off-time. Table three below defines the approximate range of parameters,

TABLE 3
Electrical parameter range delivered to the nerve

| PARAMER | RANGE |
|---|---|
| Pulse Amplitude | 0.1 Volt-10 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 2 are the electrical signals delivered to the nerve via the two electrodes 61,62 (distal and proximal) around the nerve, as shown in FIGS. 20 and 26. It being understood that the signals generated by the external pulse generator and transmitted via the primary coil 46 (antenna) are larger, because the attenuation factor between the primary coil and secondary coil is approximately 10-20 times, depending upon the distance, and orientation between the two coils. Accordingly, the range of transmitted signals of the pulse generator are approximately 10-20 times larger than shown in Table 3.

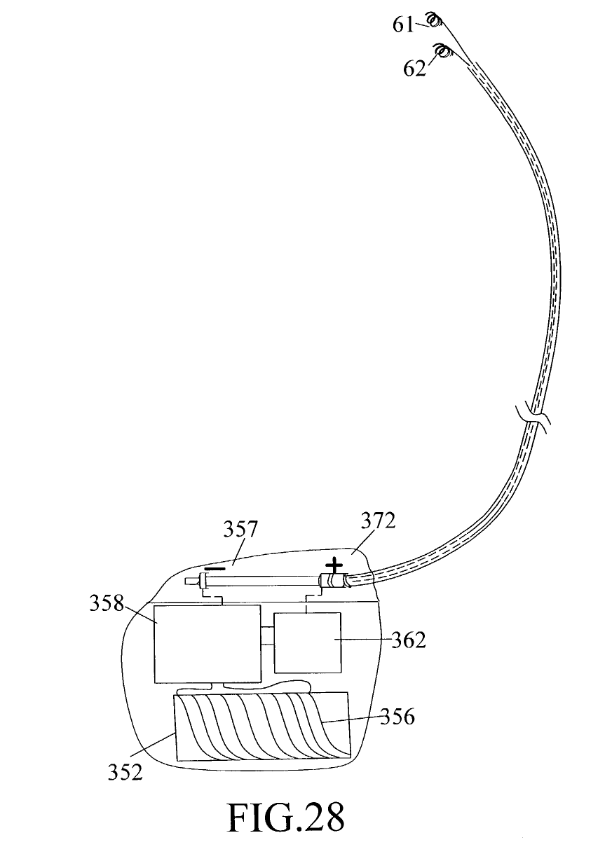

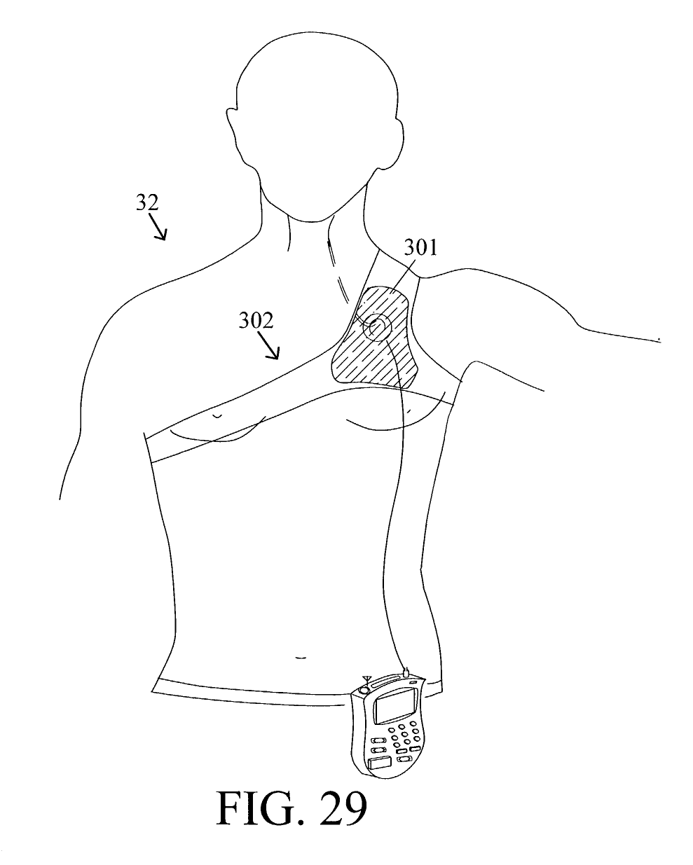

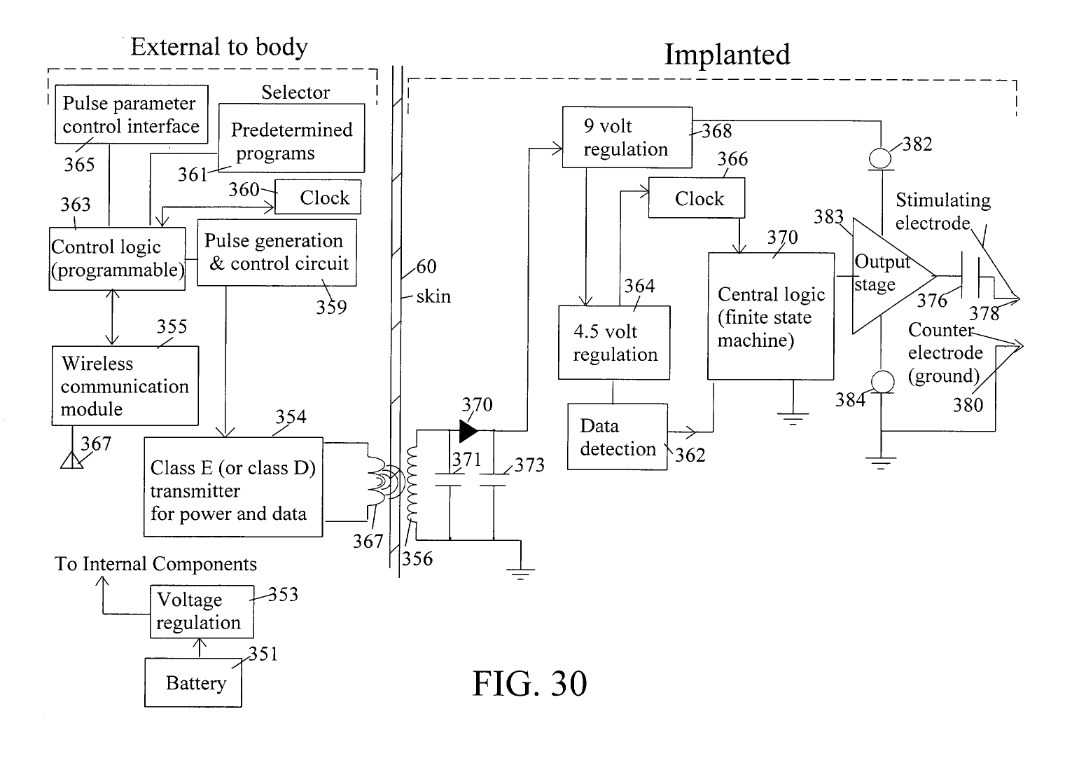

Another embodiment using the same principles is described schematically in FIGS. 28 and 30. Using mostly hybrid components and appropriate packaging, the implanted portion of the system described below is conducive to miniaturization. As shown in FIG. 28, a solenoid coil 356 wrapped around a ferrite core 352 is used as the secondary of an air gap transformer for receiving power and data to the implanted device 34. The primary coil is external to the body. Since the coupling between the external transmitter coil 367 and receiver coil 356 may be weak, a high-efficiency transmitter/amplifier is used in order to supply enough power to the receiver coil 356. Class-D or Class-E power amplifiers may be used for this purpose, and are described later. As shown in FIG. 29, the coil for the external transmitter (primary coil) may be placed in the pocket 301 of a customized garment 302.

As shown in FIG. 30, the received signal after being picked by the resonant tank circuit comprising of inductor 356 and capacitor 371, goes through a rectifier 370. Even though a single diode 370 is shown in the figure, a diode bridge can be used for full-wave rectification, and the signal then goes through two series voltage regulators in order to generate the required supply voltages. The voltage regulators consist of rectifier, storage capacitor, and 4.5-V and 9-V shunt regulators implemented using Zenor diodes and resistors (not shown in FIG. 30). Bipolar transistors and diodes with high breakdown voltages are used to provide protection from high input voltages. Clock 366 is regenerated from the radio-frequency (RF) carrier by taking the peak amplitude of sinusoidal carrier input and generating a 4.5 V square wave output. Data detection circuitry is comprised using a low-pass filter (LPF), a high-pass filter (HPF), and a Schmitt trigger for envelope detection and noise suppression. The low-pass filter is necessary in order to extract the envelope from the high frequency carrier. Finally, the output circuit contains charge-balance circuitry, stimulus current regulator circuitry, and startup circuitry.

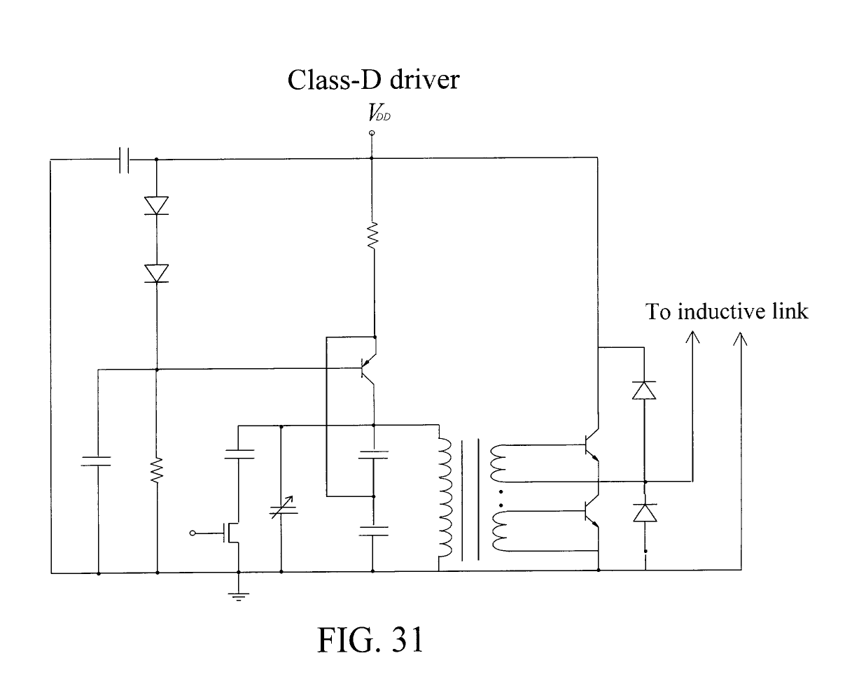

As shown in FIG. 30, a Class-D or Class E driver can be used in the external transmitter. A typical workable Class D driver is shown in FIG. 31. Class-D transmitter can drive the loads efficiently, and can supply a constant driving source so that the link output voltage, or current, remains stable. A Class-D transmitter can drive these loads efficiently because it can supply a constant source which is independent of the load. It simply switches the input of the link between the two terminals of the power supply. Reactive loads and load variation due to changing coupling should not affect its output level.

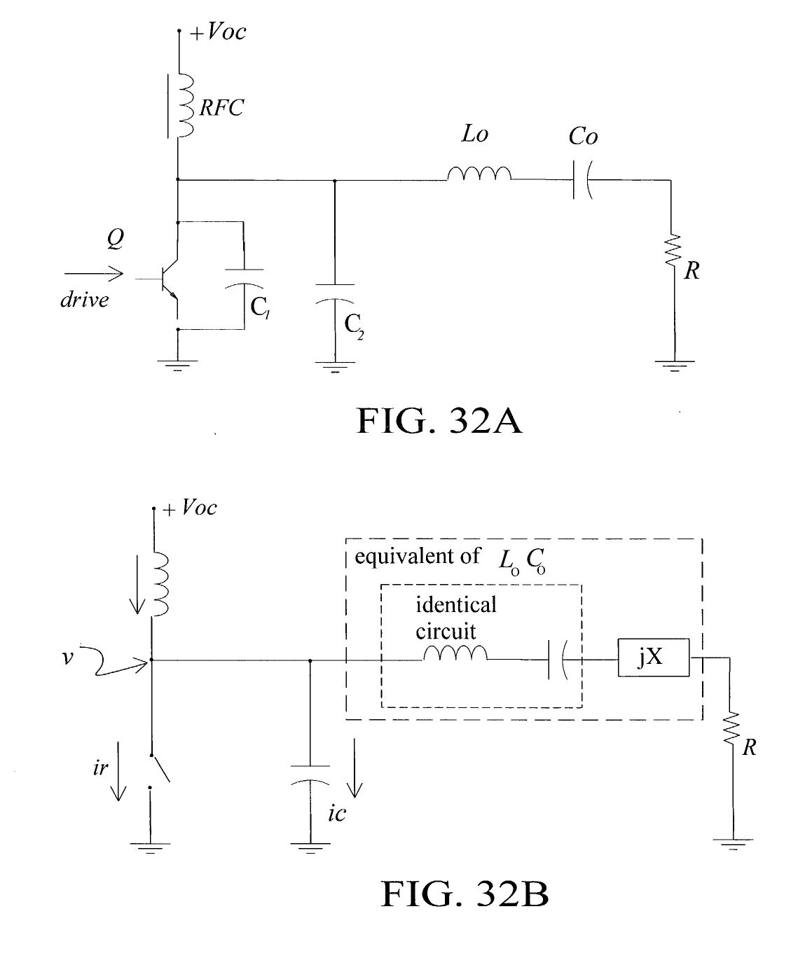

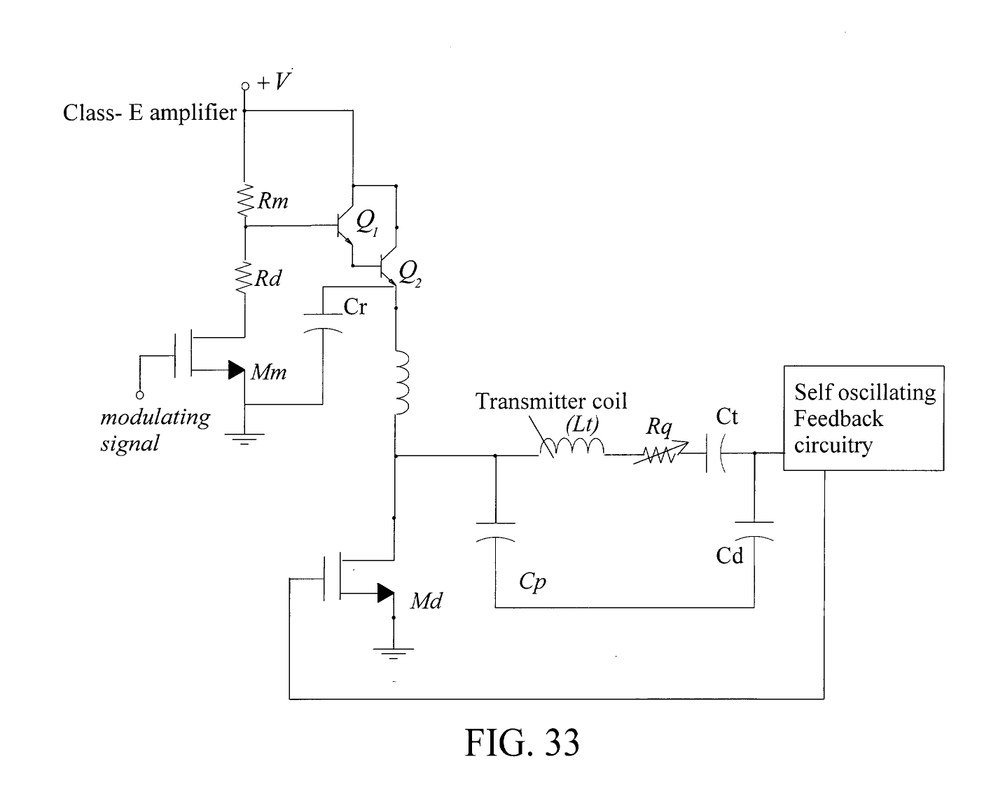

Even though both Class-D and Class-E transmitters are highly efficient, the Class-E operation of the presently preferred embodiment is explained in relation to FIGS. 32A, 32B, and 33. A basic circuit of Class-E amplifier is shown in FIG. 32A and its equivalent is shown in FIG. 32B. The “Class E” refers to a tuned power amplifier composed of a single-pole switch and a load network. The switch consists of a transistor or combination of transistors and diodes that are driven on and off at the carrier frequency of the signal to be amplified. In its most basic form, the load network consists of a resonant circuit in series with the load, and a capacitor which shunts the switch, FIGS. 32A and 32B. The total shunt capacitance is due to what is inherent in the transistor (C1) and added by the load network (C2). The collector or voltage waveform is then determined by the switch when it is on, and by the transient response of the load network when the switch is off.

In comparison, classes A, B, and C refer to amplifiers in which the transistors act as current sources; sinusoidal collector voltages are maintained by the parallel-tuned output circuit. If the transistors are driven hard enough to saturate, they cease to be current sources; however, the sinusoidal collector voltage remains. Class D is characterized by two (or more) pole switching configuration that define either a voltage current waveform without regard for the load network. Class D employs band-pass filtering. Table four below, compares the power and efficiency between different classes of amplifiers.

TABLE 4

| Class | Pₘₐₓ | Efficiency | Comments |
|---|---|---|---|
| A | 0.125 | 50% | 360° conduction angle |
| B | 0.125 | 78.5% | 180° conduction angle |
| C | 0.0981 | 89.6% | 120° conduction angle |
| D | 0.318 | 100% | uses two devices with 1 A |
| | | | peak current |
| E | 0.0981 | 100% | Optimum 50% duty cycle |

Class E power amplifiers (as well as Class D and saturating Class C power amplifiers) might more appropriately be called power converters. In these circuits, the driving signal causes switching of the transistor, but there is no relationship between the amplitudes of the driving signal and the output signal. In Class E amplifiers, there is no clear source of voltage or current, as in classes A, B, C, and D amplifiers. The collector voltage waveform is a function of the current charging the capacitor, and current is function of the voltage on the load, which is in turn a function of the collector voltage. All parameters are interrelated. A typical workable Class-E driver is shown in FIG. 33.

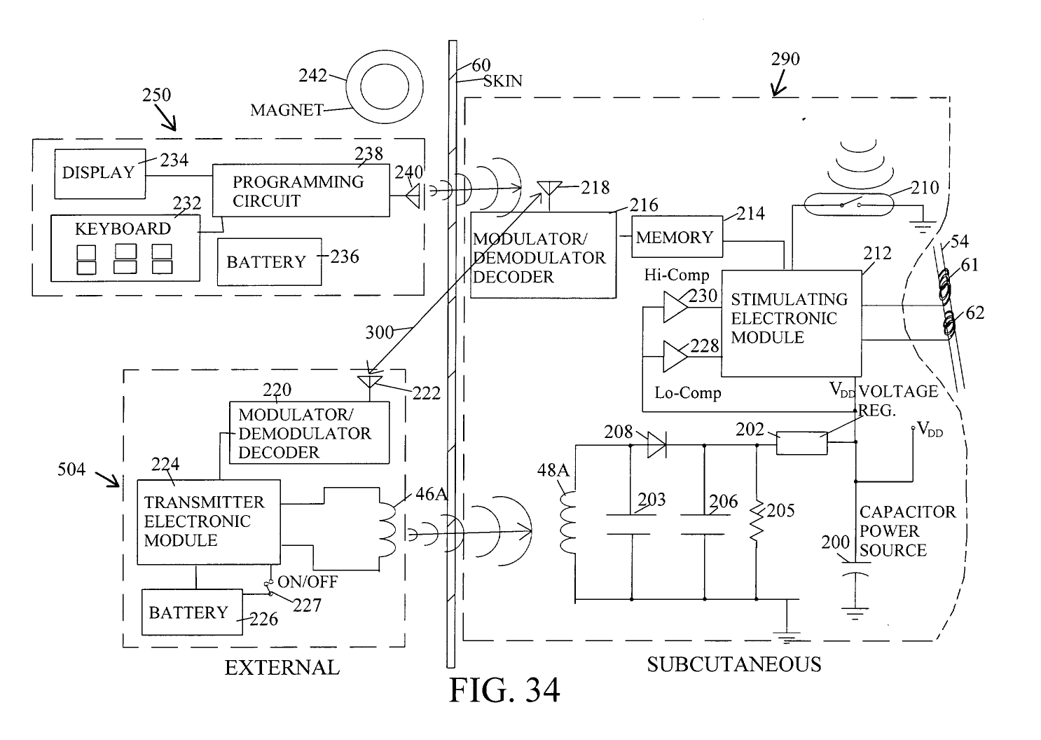

As previously mentioned, the implanted stimulus receiver may be a system which is RF coupled combined with a power source. In such a case the following embodiment may be used, where the implanted stimulator contains a high value, small sized capacitors for storing charge and delivering electric stimulation pulses for up to several hours by itself, once the capacitors are charged. As shown in FIG. 34 of the implanted stimulator 290 and the system, the receiving inductor 48A and tuning capacitor 203 are tuned to the frequency of the transmitter. The diode 208 rectifies the AC signals, and a small sized capacitor 206 is utilized for smoothing the input voltage V₁ fed into the voltage regulator 202. The output voltage V_{D} of regulator 202 is applied to capacitive energy power supply and source 200 which establishes source power V_{DD}. Capacitor 200 is a big value, small sized capacative energy source which is classified as low internal impedance, low power loss and high charge rate capacitor, such as Panasonic Model No. 641.

The refresh-recharge transmitter unit 204 includes a primary battery 226, an ON/Off switch 227, a transmitter electronic module 224, an RF inductor power coil 46A, a modulator/demodulator 220 and an antenna 222.

When the ON/OFF switch is on, the primary coil 46A is placed in close proximity to skin 60 and secondary coil 48A of the implanted stimulator. The inductor coil 46A emits RF waves establishing EMF wave fronts which are received by secondary inductor 48A. Further, transmitter electronic module 224 sends out command signals which are converted by modulator/demodulator decoder 220 and sent via antenna 222 to antenna 218 in the implanted stimulator. These received command signals are demodulated by decoder 216 and replied and responded to, based on a program in memory 214 (matched against a “command table” in the memory). Memory 214 then activates the proper controls and the inductor receiver coil 48A accepts the RF coupled power from inductor 46A.

The RF coupled power, which is alternating or AC in nature, is converted by the rectifier 208 into a high DC voltage. Small value capacitor 206 operates to filter and level this high DC voltage at a certain level. Voltage regulator 202 converts the high DC voltage to a lower precise DC voltage while capacitive power source 200 refreshes and replenishes.

When the voltage in capacative source 200 reaches a predetermined level (that is V_{DD} reaches a certain predetermined high level), the high threshold comparator 230 fires and stimulating electronic module 212 send an appropriate command signal to modulator/decoder 216. Modulator/decoder 216 then sends an appropriate “fully charged” signal indicating that capacitive power source 200 is fully charged, is received by antenna 222 in the refresh-recharge transmitter unit 204.

In one mode of operation, the patient may start or stop stimulation by waving the magnet 242 once near the implant. The magnet emits a magnetic force Lₘ which pulls reed switch 210 closed. Upon closure of reed switch 210, stimulating electronic module 212 in conjunction with memory 214 begins the delivery (or cessation as the case may be) of controlled electronic stimulation pulses to the vagus nerve via electrodes 61, 62. In another mode (AUTO), the stimulation is automatically delivered to the implanted lead based upon programmed ON/OFF times.

The programmer unit 250 includes keyboard 232, programming circuit 238, rechargable battery 236, and display 234. The physician or medical technician programs programming unit 250 via keyboard 232. This program regarding the frequency, pulse width, modulation program, ON time etc. is stored in programming circuit 238. The programming unit 250 must be placed relatively close to the implanted stimulator 290 in order to transfer the commands and programming information from antenna 240 to antenna 218. Upon receipt of this programming data, modulator/demodulator and decoder 216 decodes and conditions these signals, and the digital programming information is captured by memory 214. This digital programming information is further processed by stimulating electronic module 212. In the DEMAND operating mode, after programming the implanted stimulator, the patient turns ON and OFF the implanted stimulator via hand held magnet 242 and a reed switch 210. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

In one embodiment, the external stimulator can also have a telecommunications module, as described in a co-pending application Ser. No. 09/837565, and summarized here for reader convenience. The telecommunications module has two-way communications capabilities.

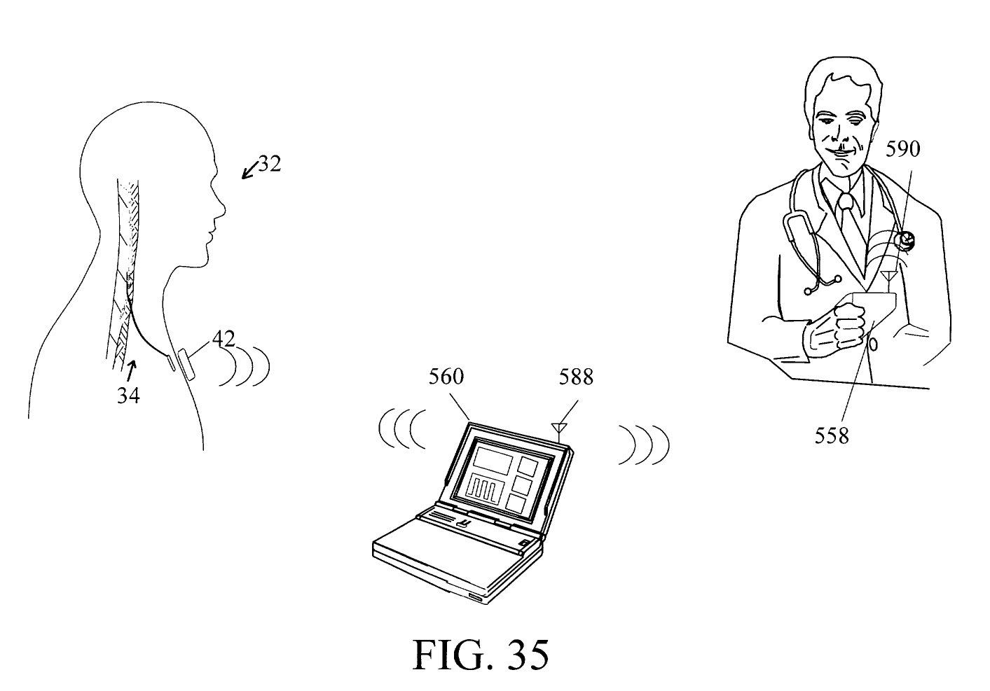

FIG. 35 shows conceptually the data communication between the external stimulator 42 and a remote hand-held computer 558. A desktop or laptop computer 560 can be a server 500 which is situated remotely, perhaps at a physician's office or a hospital. The stimulation parameter data of the stimulator, can be viewed at this facility or reviewed remotely by medical personnel on a hand-held mobile device such as personal digital assistant (PDA) 558, for example, a “palm-pilot” from PALM Corp. (Santa Clara, Calif.), a “HP Jornada” from Hewlett Pacard Corp. or on a personal computer (PC). The physician or appropriate medical personnel, is able to interrogate the external stimulator 42 device and know what the device is currently programmed to, as well as, get a graphical display of the pulse train. The wireless communication with the remote server 560 and hand-held mobile device 558 would be supported in all geographical locations within and outside the United States (US) that provides cell phone voice and data communication service. The pulse generation parameter data can also be viewed on the handheld devices (PDA) 558.

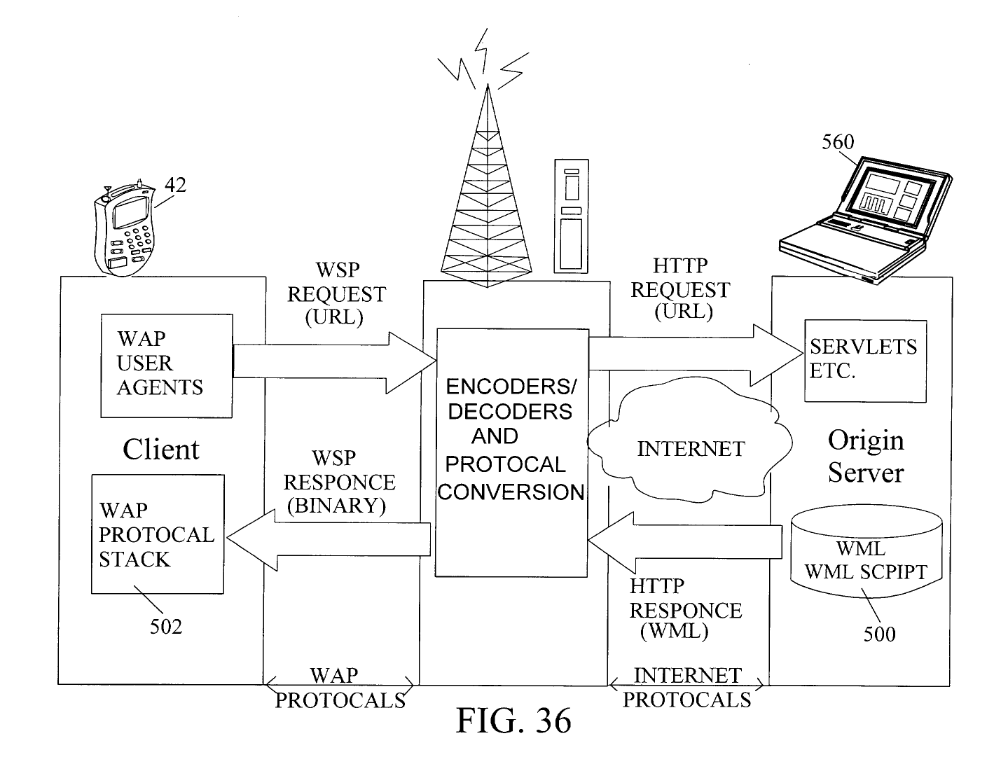

The telecommunications component of this invention uses Wireless Application Protocol (WAP). The Wireless Application Protocol (WAP) is a set of communication protocols standardizing Internet access for wireless devices. While previously, manufacturers used different technologies to get Internet on hand-held devices, with WAP devices and services interoperate. WAP promotes convergence of wireless data and the Internet. The WAP programming model is heavily based on the existing Internet programming model, and is shown schematically in FIG. 36. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops. Such features are facilitated with WAP

The key components of the WAP technology, as shown in FIG. 36, includes 1) Wireless Mark-up Language (WML) 500 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 502 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

The physician is also able to set up long-term schedules of stimulation therapy for their patient population, through wireless communication with the server. The server in turn communicates these programs to the neurostimulator 42. Each schedule is securely maintained on the server, and is editable by the physician and can get uploaded to the patient's stimulator device at a scheduled time. Thus, therapy can be customized for each individual patient. Each device issued to a patient has a unique identification key in order to guarantee secure communication between the wireless server 560 and stimulator device 42.

The second mode of communication is the ability to remotely interrogate and monitor the stimulation therapy on the physician's handheld (PDA) 558.

What is claimed is:

1. A method of selectively stimulating the vagus nerve of a patient with electrical pulses, comprising applying modulated high frequency pulses with an external pulse generator, and said pulses being received by an implanted stimulus receiver in electrical contact with said vagus nerve, and further repeatedly applying said pulses to said vagus nerve over a period of time.

2. The method of claim 1, wherein said implanted stimulus receiver does not contain a battery.

3. The method of claim 1, wherein said implanted stimulus receiver contains a battery power source and memory storage means.

4. The method of claim 1, wherein said implanted stimulus receiver contains an internal power source comprising of high value capacitive means.

5. The method of claim 1, wherein said selective stimulation does not significantly slow the heart rate.

6. The method of claim 1, wherein said external pulse generator comprises a telecommunications module.

7. The method of claim 1, wherein said external pulse generator can be remotely programmed.

8. The method of claim 1, whereby said vagus nerve is selectively stimulated for providing therapy for at least one of neurological, neuropsychiatric, and psychiatric disorders.

9. The method of claim 1, wherein said external pulse generator can be remotely interrogated.

10. The method of claim 1, wherein said selective stimulation can be controlled by pre-determined stimulation programs.

11. The method of claim 1, wherein variable parameters of said electrical pulses comprising from a group consisting, at least one of pulse amplitude, pulse width, pulses per second, on-time, and off-time; and said parameters can be individually adjusted for the required therapy.

12. The method of claim 1, wherein variable parameters of said electrical pulses can be optimized for the patient and stored in memory.

13. The method of claim 1, wherein, said high frequency carrier pulses can range in frequency from 100 Kilo-Hertz to 100 Mega-Hertz.

14. The method of claim 1 wherein, electrical stimulus pulse frequency delivered to the vagus nerve can range from 5 pulses per second to 500 pulses per second.

15. The method of claim 1 wherein, pulse amplitude delivered to the vagus nerve can range from 0.1 volts to 10 volts.

16. The method of claim 1 wherein, pulse width delivered to the vagus nerve can range from 20 micro-seconds to 5 milli-seconds.

17. The method of claim 1, wherein afferent vagus activity is modulated.

18. The method of claim 1, wherein efferent vagus activity is modulated.

19. The method of claim 1, wherein right vagus nerve is modulated.

20. The method of claim 1, wherein left vagus nerve is modulated.

21. A method of modulating the vagus nerve of a patient, comprising the steps of:
a) providing modulated high frequency electrical pulses generated and transmitted from outside the body via at least one primary coil; and
b) providing an implanted stimulus receiver comprising a secondary coil, circuitry, high value capacitve means for power storage, and at least one electrode in electrical contact with the vagus nerve,
whereby, said electrical pulses modulate the vagus nerve.

22. The method of claim 21 wherein, said high frequency carrier pulses can range in frequency from 100 Kilo-Hertz to 100 Mega-Hertz.

23. The method of claim 21 wherein, electrical stimulus pulse frequency delivered to the vagus nerve can range from 5 pulses per second to 500 pulses per second.

24. The method of claim 21 wherein, pulse amplitude delivered to the vagus nerve can range from 0.1 volts to 10 volts.

25. The method of claim 21 wherein, pulse width delivered to the vagus nerve can range from 20 micro-seconds to 5 milli-seconds.

26. The method of claim 21, wherein said therapy comprises therapy for central nervous system (CNS) disorders.

27. The method of claim 21, wherein said electrical pulses are applied repeatedly over a period of time.

28. The method of claim 21, wherein afferent vagus nerve is modulated.

29. The method of claim 21, wherein efferent vagus nerve is modulated.

30. The method of claim 21, wherein said selective stimulation does not significantly slow the heart rate.

31. The method of claim 21, wherein right vagus nerve is modulated.

32. The method of claim 21, wherein left vagus nerve is modulated.

33. A method of stimulating the vagus nerve by modulated electrical pulses, comprising the steps of:
a) providing generating means for said modulated electrical pulses;
b) providing transmitting means for transmitting said electrical pulses; and
c) providing receiving means with at least one electrode adopted to be in contact with said vagus nerve,
whereby, said vagus nerve is modulated by said electrical pulses applied repeatedly over a period of time.

34. The method of claim 33, whereby said vagus nerve is stimulated for providing therapy for at least of neurological, neuropsychiatric, and psychiatric disorders.

35. The method of claim 33, wherein said selective stimulation does not significantly slow the heart rate.

36. A system for selectively stimulating the vagus nerve without substantially slowing the heart rate comprising,
a) a pulse generator-transmitter for generating and transmitting modulated high frequency pulses, and
b) an implanted stimulus receiver in electrical contact with the vagus nerve for receiving and conditioning the electrical pulses,
whereby, neuromodulation of the vagus nerve is performed.

37. The system of claim 36, wherein said stimulation is applied repeatedly according to a predetermined program.

38. The system of claim 36, wherein said selective stimulation of the vagus nerve is performed over a period of time.

39. The system of claim 36, wherein said neuromodulation is afferent neuromodulation of the vagus nerve.

40. The system of claim 36, wherein said implanted stimulus receiver does not contain a battery.

41. The system of claim 36, wherein said implanted stimulus receiver contains a battery power source and memory device.

42. The system of claim 36, wherein said implanted stimulus receiver contains an internal power source comprising of high value capacative means.

43. The system of claim 36, wherein said pulse generator-transmitter comprises a telecommunications module.

44. The system of claim 36, wherein said pulse generator-transmitter can be remotely programmed.

45. The system of claim 36, whereby said vagus nerve is selectively stimulated for providing therapy for at least one of neurological, neuropsychiatric, and psychiatric disorders.

46. The system of claim 36, wherein said external pulse generator can be remotely interrogated.

47. The system of claim 36, wherein said electrical pulses comprises variable components consisting from a group comprising, pulse amplitude, pulse width, pulse frequency, on-time, and off-time.

48. A system for modulating the vagus nerve of a patient for providing therapy comprising:
a) a pulse generator for generating electrical pulses, and at least one primary coil for transmitting high frequency modulated pulses; and
b) an implanted stimulus receiver comprising a secondary coil, circuitry, high value capacitive means for power storage, and at least one electrode in electrical contact with the vagus nerve,
said modulated pulses, received by said secondary coil are delivered to the vagus nerve for providing said therapy.

49. The system of claim 48, wherein said therapy comprises therapy for at least one of neurological, neuropsychiatric, and psychiatric disorders.

50. The system of claim 48, wherein said vagus nerve modulation is performed over a period of time.

51. The system of claim 48, wherein said secondary coil of said implanted stimulus receiver comprises a solenoid coil wrapped around a ferrite core.

52. The system of claim 48, wherein said primary coil and said secondary coil are pancake shaped flat coils.

53. The system of claim 48, wherein said implanted stimulus receiver does not contain a battery.

54. The system of claim 48, wherein said implanted stimulus receiver contains a battery power source and memory device.

55. The system of claim 48, wherein said electrical pulses comprises variable parameters consisting from a group comprising, pulse amplitude, pulse width, pulse frequency, on-time, and off-time.

56. The system of claim 48, wherein said external pulse generator comprises a telecommunications module.

57. The system of claim 48, wherein said external pulse generator can be remotely programmed.

58. The system of claim 48, wherein said external pulse generator can be remotely interrogated.
